(19) United States
(12) Patent Application Publication
(10) Pub. No.: US 2026/0069559 A1
(43) Pub. Date: Mar. 12, 2026

(54) COMBINATIONS OF RXFP1 MODULATORS AND SGLT2 INHIBITORS

(71) Applicant: ASTRAZENECA AB, Sodertalje (SE)

(72) Inventors: Anders Gabrielsen, Sodertalje (SE); Daniel Tor Pettersen, Sodertalje (SE); Kathleen Marie Connolly, Cambridge (GB); Marcin Ufnal, Warszawa (PL)

(21) Appl. No.: 18/872,319

(22) PCT Filed: Jun. 6, 2023
(86) PCT No.: PCT/EP2023/065046

§ 371 Date: Dec. 6, 2024

Related U.S. Application Data
(60) Provisional application No. 63/365,969, filed on Jun. 7, 2022.

(51) Int. Cl. A61K 31/196; A61K 31/70

(52) U.S. Cl. CPC A61K 31/196; A61K 31/70

(57) ABSTRACT

The specification generally relates to combinations of RXFP1 modulators and SGLT2 inhibitors, in particular pharmaceutical compositions comprising such combinations, and therapeutic methods using such combinations.

### FIELD

Described in this specification are combinations of compounds (including salts thereof) that are modulators of RXFP1, and SGLT2 inhibitors such as dapagliflozin; pharmaceutical compositions comprising such combinations and uses of such combinations.

### BACKGROUND

Relaxin is a pleiotropic hormone known to mediate systemic haemodynamic and renal adaptive changes during pregnancy. Relaxin has also been shown to have anti-fibrotic properties and to have beneficial effects in heart failure e.g. with acute decompensated heart failure (ADHF). Heart failure is associated with significant morbidity and mortality. It is characterized by complex tissue remodelling involving increased cardiomyocyte death and interstitial fibrosis. Relaxin activates a number of signalling cascades which have been shown to be beneficial in the setting of ischemia-reperfusion and heart failure. These signalling pathways include activation of the phosphoinositide 3-kinase pathway and activation of the nitric oxide signalling pathway (Bathgate R A et al. (2013) Physiol. Rev. 93(1): 405-480; Mentz R J et al. (2013) Am. Heart J 165(2): 193-199; Tietjens J et al. (2016) Heart 102: 95-99; Wilson S S et al. (2015) Pharmacology 35: 315-327).

In heart failure patients, a significant subset also suffer from pulmonary hypertension (HF+PH patients). It was estimated that approximately 50% of heart failure patients with preserved ejection fraction also suffer from pulmonary hypertension, increasing to 60% of heart failure patients with reduced ejection fraction (Guazzi, (2014) Circ Heart Fail., 7:367-377; Miller et al., (2013) JACC Heart Fail., 1(4):290-299). Patients suffering from heart failure with pulmonary hypertension have been shown to have reduced survival as compared with heart failure patients without pulmonary hypertension (Barnett and De Marco, (2012) Heart Fail. Clin. 8: 447-459). In heart failure patients, a 3 mmHg increase or decrease in Estimated Pulmonary Artery Diastolic Pressure (ePAD), equivalent to approximately 4 mmHg increase or decrease in mean Pulmonary Arterial Pressure (mPAP), was associated with a 24% increase or a 19% decrease in cardiovascular mortality respectively (Zile M R, et al. (2017) Circ Heart Fail., 10:e003594). A 4 mmHg reduction in mPAP is also associated with dyspnea improvement in patients suffering from heart failure and pulmonary hypertension (Solomonica A, et al. (2013) Circ Heart Fail., 6:53-60).

Resistant hypertension (rHT) is defined as the blood pressure of a hypertensive patient that remains elevated above target goal despite the concurrent use of optimized doses of 3 antihypertensive agents of different classes, one of which is a diuretic. Current SoC for the initial treatment of hypertension is a calcium channel blocker (CCB), a blocker of the renin-angiotensin system (angiotensin-converting enzyme [ACE] inhibitor or angiotensin receptor blocker [ARB]), and a diuretic. For patients with rHT, there are multiple options for what to add next (such as a mineralocorticoid-receptor antagonist (MRA), beta-blocker, or alpha-blocker) and guidelines currently recommend a MRA as preferred option for treatment of rHT. rHT also includes patients whose blood pressure is adequately controlled when receiving 4 or more antihypertensive medications concurrently (Carey et al., Hypertension, 2018, 72, e53-e90). Patients with rHT typically have long histories of severe blood pressure elevation, predisposing them to higher cardiovascular risk than treated hypertensive patients with controlled blood pressure (Acelajado et al., Circulation Research, 2019, 124, 1061-1070). It has been suggested that relaxin may have therapeutic potential for hypertensive disease (Lekgabe et al., Hypertension, 2005, 46, 412-8).

Clinical trials have been conducted using unmodified recombinant human Relaxin-2, serelaxin. Continuous intravenous administration of serelaxin to hospitalized patients improved the markers of cardiac, renal and hepatic damage and congestion (Felker G M et al. (2014) J Am. Coll. Cardiol. 64(15): 1591-1598; Metra M et al. (2013) J Am. Coll. Cardiol. 61(2): 196-206; Teerlink J R et al. (2013) Lancet 381(9860): 29-39). However, due to the rapid clearance of serelaxin from the patients' circulation, the therapeutic effects were limited and the positive effects rapidly disappeared once intravenous injection stopped. Additionally, approximately one third of the patients experienced a significant blood pressure drop (>40 mm Hg) after receiving serelaxin intravenously, with the consequence that the dose had to be reduced by half or even more.

The cognate receptor for human relaxin is RXFP1 and is a well-validated pharmacologically important GPCR family 1c member whose activation by the hormone relaxin is associated with hemodynamic, anti-fibrotic and anti-inflammatory properties (Halls M L et al., (2015), Pharmacol Rev. 67(2): 389-440).

Small-molecule modulators of RXFP1 have been sought as relaxin mimetics. For example, Marugan, J. J., et al., WO2013/165606A1; Xiao J et al. (2013) Nat. Commun. 4:1953; and McBride A et al. (2017) Scientific Reports 7:10806 discuss small-molecule modulators of RXFP1.

Despite the foregoing, a need continues to exist for further compounds that are modulators of RXFP1 which may make the compounds especially promising for development as therapeutic agents. Such compound(s) may also exhibit improved modulation of RXFP1 in comparison with other known RXFP1 modulators. Such compound(s) may also exhibit favourable pharmacokinetic profiles (for example, lower intrinsic clearance) and/or advantageous physical properties (for example, higher aqueous solubility) in comparison with other known RXFP1 modulators. Therefore, such compound(s) may be especially useful in the treatment of disease states in which modulation of RXFP1 is beneficial. Furthermore, combinations of such compound(s) with other therapeutic agents may be useful in the treatment of certain conditions such as hypertension.

### SUMMARY

The specification relates to combinations of RXFP1 modulators described herein with SGLT2 inhibitors. SGLT2 inhibitors such as dapagliflozin have been shown to exhibit blood pressure-lowering effects (Weber et al., Lancet Diabetes Endocrinol., 2016, 4(3), 211-220). Accordingly, combinations of RXFP1 modulators with SGLT2 inhibitors may provide particular benefit in the treatment of hypertension, in particular resistant hypertension, and cardiovascular diseases for which there remains a significant unmet need.

Accordingly, this specification describes, in part, an RXFP1 modulator for use in therapy of a human patient, wherein the RXFP1 modulator is administered in combination with an SGLT2 inhibitor, and wherein the RXFP1 modulator is selected from:

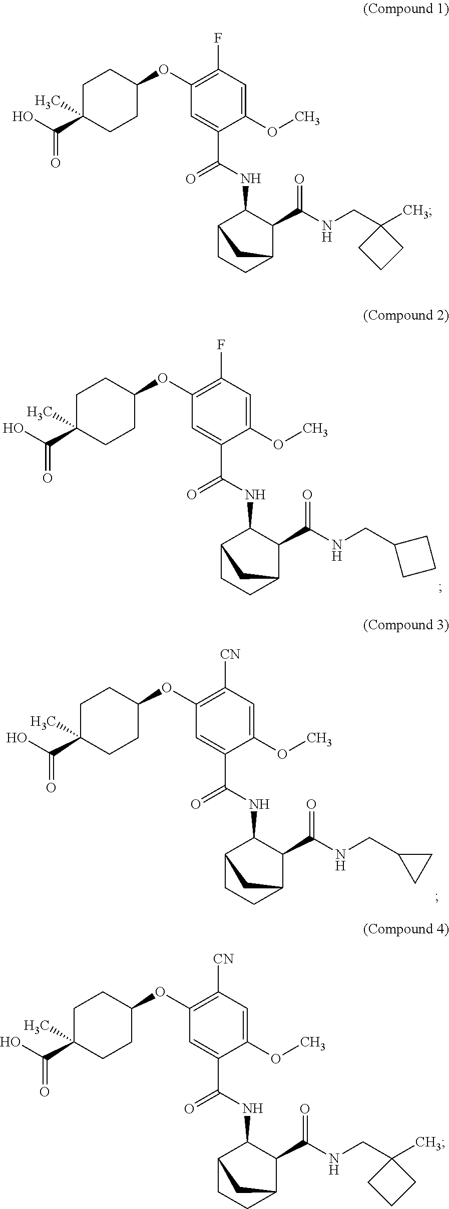 [SMILES: COc1cc(F)c(O[C@H]2CC[C@@](C)(C(=O)O)CC2)cc1C(=O)N[C@@H]1[C@H]2CC[C@H](C2)[C@@H]1C(=O)NCC1(C)CCC1 ; COc1cc(F)c(O[C@H]2CC[C@@](C)(C(=O)O)CC2)cc1C(=O)N[C@@H]1[C@H]2CC[C@H](C2)[C@@H]1C(=O)NCC1CCC1 ; COc1cc(C#N)c(O[C@H]2CC[C@@](C)(C(=O)O)CC2)cc1C(=O)N[C@@H]1[C@H]2CC[C@H](C2)[C@@H]1C(=O)NCC1CC1 ; COc1cc(C#N)c(O[C@H]2CC[C@@](C)(C(=O)O)CC2)cc1C(=O)N[C@@H]1[C@H]2CC[C@H](C2)[C@@H]1C(=O)NCC1(C)CCC1]

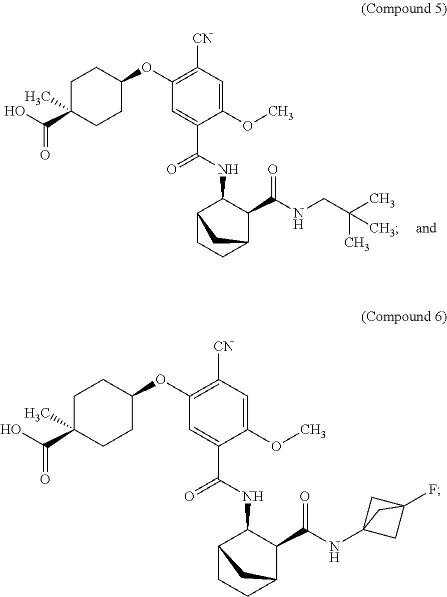 [SMILES: COc1cc(C#N)c(O[C@H]2CC[C@@](C)(C(=O)O)CC2)cc1C(=O)N[C@@H]1[C@H]2CC[C@H](C2)[C@@H]1C(=O)NCC(C)(C)C ; COc1cc(C#N)c(O[C@H]2CC[C@@](C)(C(=O)O)CC2)cc1C(=O)N[C@@H]1[C@H]2CC[C@H](C2)[C@@H]1C(=O)NC12CC(F)(C1)C2]

or a pharmaceutically acceptable salt thereof.

Similarly, this specification describes, in part, an RXFP1 modulator for use in the treatment of a condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient, wherein the RXFP1 modulator is administered in combination with an SGLT2 inhibitor, and wherein the RXFP1 modulator is selected from: Compound 1, Compound 2, Compound 3, Compound 4, Compound 5 and Compound 6, or a pharmaceutically acceptable salt thereof.

Similarly, this specification describes, in part, a method of treating a condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant is hypertension in a human patient in need of such treatment, comprising administering to the human patient a therapeutically effective amount of an RXFP1 modulator, wherein the RXFP1 modulator is administered in combination with a therapeutically effective amount of an SGLT2 inhibitor, and wherein the RXFP1 modulator is selected from: Compound 1, Compound 2, Compound 3, Compound 4, Compound 5 and Compound 6, or a pharmaceutically acceptable salt thereof.

Similarly, this specification describes, in part, a method of treating a condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient in need of such treatment, comprising administering to the human patient a first amount of an RXFP1 modulator, and a second amount of an SGLT2 inhibitor, where the first amount and the second amount together comprise a therapeutically effective amount, and wherein the RXFP1 modulator is selected from: Compound 1, Compound 2, Compound 3, Compound 4, Compound 5 and Compound 6, or a pharmaceutically acceptable salt thereof.

Similarly, this specification describes, in part, a pharmaceutical composition comprising an RXFP1 modulator, an SGLT2 inhibitor, and a pharmaceutically acceptable excipient, and wherein the RXFP1 modulator is selected from: Compound 1, Compound 2, Compound 3, Compound 4, Compound 5 and Compound 6, or a pharmaceutically acceptable salt thereof.

Similarly, this specification describes, in part, a kit comprising:
a first pharmaceutical composition comprising an RXFP1 modulator selected from: Compound 1, Compound 2, Compound 3, Compound 4, Compound 5 and Compound 6, or a pharmaceutically acceptable salt thereof, and a pharmaceutically acceptable excipient; and
a second pharmaceutical composition comprising an SGLT2 inhibitor and a pharmaceutically acceptable excipient.

Further aspects of the disclosure will be apparent to one skilled in the art from reading this specification.

### DESCRIPTION OF ILLUSTRATIVE EMBODIMENTS

Many embodiments are detailed throughout the specification and will be apparent to a reader skilled in the art. The specification is not to be interpreted as being limited to any particular embodiment(s) described herein.

Terms not specifically defined herein should be understood to have the meanings that would be given to them by one of skill in the art in light of the disclosure and the context.

“About” may generally mean an acceptable degree of error for the quantity measured given the nature or precision of the measurements. Exemplary degrees of error are within percent (%), typically, within 10%, and more typically, within 5% of a given value or range of values.

Embodiments described herein as “comprising” one or more features may also be considered as disclosure of the corresponding embodiments “consisting of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

The chemical names of compounds described in this specification were generated using ChemDraw® Professional version 19.0.0.22 from PerkinElmer®. The skilled person will understand that different chemical naming software may generate different chemical names for a particular compound. In case a compound described herein is depicted in form of a chemical name and as a formula, the formula shall prevail in case of any discrepancy.

### RXFP1 Modulators

In one embodiment the RXFP1 modulator is selected from:

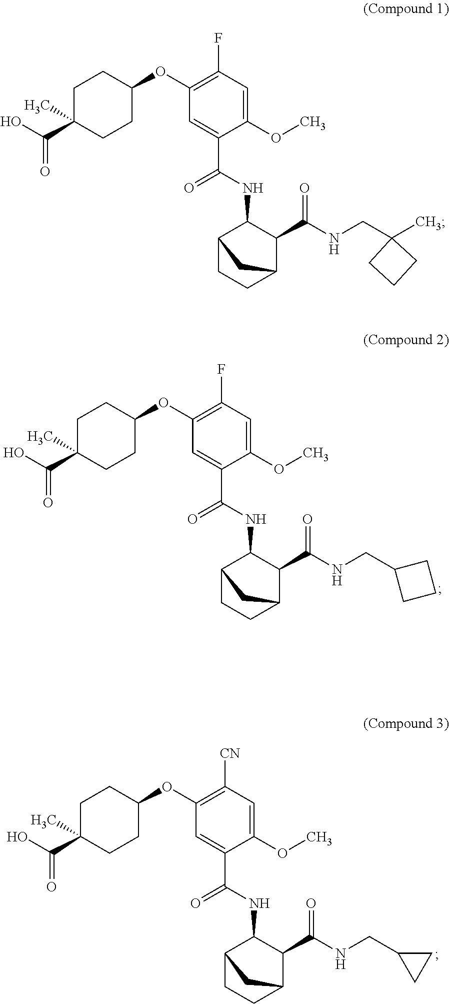 [SMILES: COc1cc(F)c(O[C@H]2CC[C@@](C)(C(=O)O)CC2)cc1C(=O)N[C@@H]1[C@H]2CC[C@H](C2)[C@@H]1C(=O)NCC1(C)CCC1 ; COc1cc(F)c(O[C@H]2CC[C@@](C)(C(=O)O)CC2)cc1C(=O)N[C@@H]1[C@H]2CC[C@H](C2)[C@@H]1C(=O)NCC1CCC1 ; COc1cc(C#N)c(O[C@H]2CC[C@@](C)(C(=O)O)CC2)cc1C(=O)N[C@@H]1[C@H]2CC[C@H](C2)[C@@H]1C(=O)NCC1CC1]

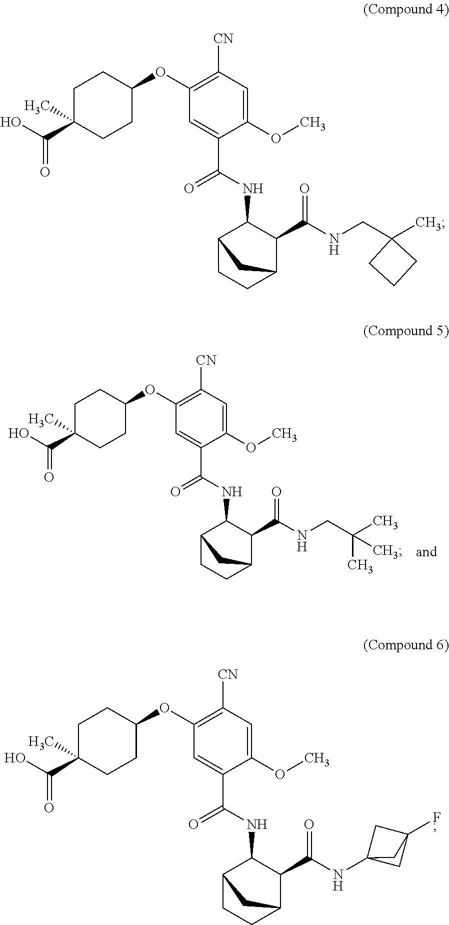 [SMILES: COc1cc(C#N)c(O[C@H]2CC[C@@](C)(C(=O)O)CC2)cc1C(=O)N[C@@H]1[C@H]2CC[C@H](C2)[C@@H]1C(=O)NCC1(C)CCC1 ; COc1cc(C#N)c(O[C@H]2CC[C@@](C)(C(=O)O)CC2)cc1C(=O)N[C@@H]1[C@H]2CC[C@H](C2)[C@@H]1C(=O)NCC(C)(C)C ; COc1cc(C#N)c(O[C@H]2CC[C@@](C)(C(=O)O)CC2)cc1C(=O)N[C@@H]1[C@H]2CC[C@H](C2)[C@@H]1C(=O)NC12CC(F)(C1)C2]

or a pharmaceutically acceptable salt thereof.

In one embodiment, the RXFP1 modulator is Compound 1 or a pharmaceutically acceptable salt thereof.

In one embodiment, the RXFP1 modulator is Compound 2 or a pharmaceutically acceptable salt thereof.

In one embodiment, the RXFP1 modulator is Compound 3 or a pharmaceutically acceptable salt thereof.

In one embodiment, the RXFP1 modulator is Compound 4 or a pharmaceutically acceptable salt thereof.

In one embodiment, the RXFP1 modulator is Compound 5 or a pharmaceutically acceptable salt thereof.

In one embodiment, the RXFP1 modulator is Compound 6 or a pharmaceutically acceptable salt thereof.

In one embodiment, the RXFP1 modulator is Compound 1.

In one embodiment, the RXFP1 modulator is Compound 2.

In one embodiment, the RXFP1 modulator is Compound 3.

In one embodiment, the RXFP1 modulator is Compound 4.

In one embodiment, the RXFP1 modulator is Compound 5.

In one embodiment, the RXFP1 modulator is Compound 6.

In one embodiment, the RXFP1 modulator is selected from:
(1S,4s)-4-(2-fluoro-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid;
(1S,4s)-4-(2-cyano-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid;
(1S,4s)-4-(2-Cyano-5-(((1S,2R,3S,4R)-3-((cyclopropylmethyl)carbamoyl)bicyclo[2.2.1]heptan-2-yl)carbamoyl)-4-methoxyphenoxy)-1-methylcyclohexane-1-carboxylic acid;
(1S,4s)-4-(2-cyano-4-methoxy-5-(((1S,2R,3S,4R)-3-(neopentylcarbamoyl)bicyclo[2.2.1]heptan-2-yl)carbamoyl)phenoxy)-1-methylcyclohexane-1-carboxylic acid;
(1S,4s)-4-(2-cyano-5-(((1S,2R,3S,4R)-3-((3-fluorobicyclo[1.1.1]pentan-1-yl)carbamoyl)bicyclo[2.2.1]heptan-2-yl)carbamoyl)-4-methoxyphenoxy)-1-methylcyclohexane-1-carboxylic acid; and
(1S,4s)-4-(5-(((1S,2R,3S,4R)-3-((cyclobutylmethyl)carbamoyl)bicyclo[2.2.1]heptan-2-yl)carbamoyl)-2-fluoro-4-methoxyphenoxy)-1-methylcyclohexane-1-carboxylic acid;
or a pharmaceutically acceptable salt thereof.

In one embodiment, the RXFP1 modulator is a compound as claimed or exemplified in International Patent Application No. PCT/EP2021/084673 or U.S. patent application Ser. No. 17/457,953 (both applications are incorporated by reference in their entirety).

The term “pharmaceutically acceptable” is used to specify that an object (for example a salt, dosage form or excipient) is suitable for use in patients. An example list of pharmaceutically acceptable salts can be found in the Handbook of Pharmaceutical Salts: Properties, Selection and Use, P. H. Stahl and C. G. Wermuth, editors, Weinheim/Zürich:Wiley-VCH/VHCA, 2002. A suitable pharmaceutically acceptable salt of a compound described herein is, for example, an acid-addition salt or a base-addition salt. An acid addition salt of a compound described herein may be formed by bringing the compound into contact with a suitable inorganic or organic acid under conditions known to the skilled person. An acid addition salt may for example be formed using an inorganic acid selected from the group consisting of hydrochloric acid, hydrobromic acid, sulphuric acid and phosphoric acid. An acid addition salt may also be formed using an organic acid selected from the group consisting of trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid and para-toluenesulfonic acid.

Therefore, in one embodiment an RXFP1 modulator is a pharmaceutically acceptable salt, where the pharmaceutically acceptable salt is a hydrochloric acid, hydrobromic acid, sulphuric acid, phosphoric acid, trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid or para-toluenesulfonic acid salt.

Compounds described in this specification may form base addition salts. A base-addition salt of a compound described herein may be formed by bringing the compound into contact with a suitable inorganic or organic base under conditions known to the skilled person. For example, it may be possible to make an alkali metal (such as sodium, potassium, or lithium) or an alkaline earth metal (such as a calcium) salt by treating a compound with an alkali metal or alkaline earth metal hydroxide or alkoxide (e.g., an ethoxide or methoxide) or a suitably basic organic amine (e.g., a choline or meglumine) in an aqueous medium. Therefore, in one embodiment an RXFP1 modulator is a pharmaceutically acceptable salt, where the pharmaceutically acceptable salt is a sodium, potassium, lithium, calcium, choline or meglumine salt.

Compounds and salts described in this specification may exist in solvated forms and unsolvated forms. For example, a solvated form may be a hydrated form, such as a hemi-hydrate, a mono-hydrate, a di-hydrate, a tri-hydrate or an alternative quantity thereof. All such solvated and unsolvated forms of compounds described herein are encompassed herein.

Atoms of the compounds and salts described in this specification may exist as their isotopes. All compounds described herein where an atom is replaced by one or more of its isotopes (for example a compound described herein where one or more carbon atom is an ¹¹C or ¹³C carbon isotope, or where one or more hydrogen atoms is a ²H or ³H isotope) are encompassed herein.

Compounds described herein may exist in one or more geometrical, optical, enantiomeric, and diastereomeric forms, including, but not limited to, cis- and trans-forms, E- and Z-forms, and R-, S- and meso-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods.

The compounds described herein may include one or more chiral centres. To the extent a structure or chemical name in this specification does not indicate chirality, the structure or name is intended to encompass any single stereoisomer corresponding to that structure or name, as well as any mixture of stereoisomers (e.g. a racemate). Where a structure in this specification includes bonds drawn as solid and hashed wedges (i.e. [symbol] and [symbol] ), it is intended that the solid and hashed wedges indicate the absolute configuration of a chiral centre.

It is well-known in the art how such optically-active forms can be separated. For example, a single stereoisomer can be obtained by isolating it from a mixtures of isomers (e.g. a racemate) using, for example, chiral chromatographic separation. In other embodiments, a single stereoisomer is obtained through direct synthesis from, for example, a chiral starting material.

According to one embodiment, an RXFP1 modulator is provided as a single enantiomer being in enantiomer excess (% ee) of ≥95%, ≥98%, or ≥99%. Conveniently a single enantiomer is present in an enantiomer excess of ≥99%.

According to one embodiment, an RXFP1 modulator is provided as a single enantiomer being in enantiomer excess (% ee) in the range 95 to 100%.

Compounds described herein may exist in one or more tautomeric forms, including, but not limited to, keto-, and enol-forms. A reference to a particular compound includes all tautomeric forms, including mixtures thereof. Accordingly, a structure depicted herein as one tautomer is intended to also include other tautomers.

The RXFP1 modulators described herein may be administered in the form of a prodrug, which is a compound that is broken down in the human or animal body to release such an RXFP1 modulator. Such, pharmaceutically acceptable, prodrugs of RXFP1 modulators also form an embodiment. Various forms of prodrugs are known in the art. For example, see
a) Design of Pro-drugs, edited by H. Bundgaard, (Elsevier, 1985);
b) A Textbook of Drug Design and Development, edited by Krogsgaard-Larsen and H. Bundgaard, Chapter 5 “Design and Application of Pro-drugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem. Pharm. Bull., 32, 692 (1984).

### Sodium-Glucose Transport Protein, Subtype 2 (SGLT2) Inhibitors

In some instances, glucose reabsorption in the kidney is regulated by a member of the sodium glucose cotransporter family, which are sodium-dependent glucose transport proteins. In some instances, glucose reabsorption in the kidney is regulated by sodium-glucose transport protein, subtype 2 (SGLT2).

In one embodiment, the SGLT2 inhibitor is a gliflozin. In one embodiment, the SGLT2 is inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof. In one embodiment, the SGLT2 inhibitor is selected from dapagliflozin, canagliflozin, and empagliflozin, or a pharmaceutically acceptable salt thereof. In one embodiment, the SGLT2 inhibitor is dapagliflozin, which has the following chemical structure:

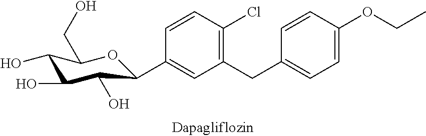

Dapagliflozin is a potent, highly selective, and orally active inhibitor of human renal sodium-dependent glucose transporter 2 (SGLT2) (SGLT2i) that has been approved to improve glycemic control in adults with type 2 diabetes mellitus (as an adjunct to diet and exercise) and to reduce the risk of hospitalization for heart failure in adults with type 2 diabetes mellitus and established cardiovascular disease or multiple cardiovascular risk factors. Dapagliflozin has been disclosed in WO2003/099836 along with details on the chemical synthesis.

### Treatment Using RXFP1 Modulators and SGLT2 Inhibitors

Combinations comprising RXFP1 modulators and SGLT2 inhibitors described herein are expected to be useful in therapy.

The term “therapy” is intended to have its normal meaning of dealing with a disease or condition in order to entirely or partially relieve one, some or all of its symptoms, or to correct or compensate for the underlying pathology. The term “therapy” also includes “prophylaxis” unless there are specific indications to the contrary. The terms “therapeutic” and “therapeutically” should be interpreted in a corresponding manner.

The term “prophylaxis” is intended to have its normal meaning and includes primary prophylaxis to prevent the development of the disease or condition and secondary prophylaxis whereby the disease or condition has already developed and the patient is temporarily or permanently protected against exacerbation or worsening of the disease or condition, or the development of new symptoms associated with the disease or condition.

The term “treatment” is used synonymously with “therapy”. Similarly the term “treat” can be regarded as “applying therapy” where “therapy” is as defined herein.

The term “therapeutically effective amount” refers to an amount of an RXFP1 modulator as described herein which is effective to provide “therapy” in a subject, or to “treat” a disease or condition in a subject. The therapeutically effective amount may cause any of the changes observable or measurable in a subject as described in the definition of “therapy”, “treatment” and “prophylaxis” above. As recognized by those skilled in the art, effective amounts may vary depending on route of administration, excipient usage, and co-usage with other agents. For example, where a combination therapy is used, the amount of the RXFP1 modulator and the amount of the other pharmaceutically active agent(s) are, when combined, jointly effective to treat a targeted disorder or condition in the subject. In this context, the combined amounts are in a “therapeutically effective amount” if they are, when combined, sufficient to decrease the symptoms of a disease or condition responsive to modulation and/or agonism of RXFP1 as described above. Typically, such amounts may be determined by one skilled in the art.

As used herein, the terms “subject” and “patient” are used interchangeably. “Subjects” include, for example, mammals, for example, humans. In some embodiments, the subject is human.

Accordingly, in one embodiment, there is provided an RXFP1 modulator for use in therapy of a human patient, wherein the RXFP1 modulator is administered in combination with an SGLT2 inhibitor, and wherein the RXFP1 modulator is selected from: Compound 1, Compound 2, Compound 3, Compound 4, Compound 5 and Compound 6, or a pharmaceutically acceptable salt thereof. In one embodiment, the RXFP1 modulator is Compound 1 or a pharmaceutically acceptable salt thereof. In one embodiment, the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof. In one embodiment, the SGLT2 inhibitor is dapagliflozin.

In one embodiment, there is provided an RXFP1 modulator for use in the treatment of a condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient, wherein the RXFP1 modulator is administered in combination with an SGLT2 inhibitor, and wherein the RXFP1 modulator is selected from: Compound 1, Compound 2, Compound 3, Compound 4, Compound 5 and Compound 6, or a pharmaceutically acceptable salt thereof. In one embodiment, the RXFP1 modulator is Compound 1 or a pharmaceutically acceptable salt thereof. In one embodiment, the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof. In one embodiment, the SGLT2 inhibitor is dapagliflozin. In one embodiment, the condition is hypertension. In one embodiment, the condition is resistant hypertension. In one embodiment, the administration of the RXFP1 modulator and the SGLT2 inhibitor is separate, sequential, or simultaneous.

In one embodiment, is provided a method of treating a condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient in need of such treatment, comprising administering to the human patient a therapeutically effective amount of an RXFP1 modulator, wherein the RXFP1 modulator is administered in combination with a therapeutically effective amount of an SGLT2 inhibitor, and wherein the RXFP1 modulator is selected from: Compound 1, Compound 2, Compound 3, Compound 4, Compound 5 and Compound 6, or a pharmaceutically acceptable salt thereof. In one embodiment, the RXFP1 modulator is Compound 1 or a pharmaceutically acceptable salt thereof. In one embodiment, the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof. In one embodiment, the SGLT2 inhibitor is dapagliflozin. In one embodiment, the condition is hypertension. In one embodiment, the condition is resistant hypertension. In one embodiment, the administration of the RXFP1 modulator and the SGLT2 inhibitor is separate, sequential, or simultaneous.

In one embodiment, is provided a method of treating a condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient in need of such treatment, comprising administering to the human patient a first amount of an RXFP1 modulator, and a second amount of an SGLT2 inhibitor, where the first amount and the second amount together comprise a therapeutically effective amount, and wherein the RXFP1 modulator is selected from: Compound 1, Compound 2, Compound 3, Compound 4, Compound 5 and Compound 6, or a pharmaceutically acceptable salt thereof. In one embodiment, the RXFP1 modulator is Compound 1 or a pharmaceutically acceptable salt thereof. In one embodiment, the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof. In one embodiment, the SGLT2 inhibitor is dapagliflozin. In one embodiment, the condition is hypertension. In one embodiment, the condition is resistant hypertension. In one embodiment, the administration of the RXFP1 modulator and the SGLT2 inhibitor is separate, sequential, or simultaneous.

In one embodiment, there is provided an RXFP1 modulator for use in the manufacture of a medicament for the treatment of a condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient, wherein the treatment comprises administering the RXFP1 modulator in combination with an SGLT2 inhibitor, and wherein the RXFP1 modulator is selected from: Compound 1, Compound 2, Compound 3, Compound 4, Compound 5 and Compound 6, or a pharmaceutically acceptable salt thereof. In one embodiment, the RXFP1 modulator is Compound 1 or a pharmaceutically acceptable salt thereof. In one embodiment, the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof. In one embodiment, the SGLT2 inhibitor is dapagliflozin. In one embodiment, the condition is hypertension. In one embodiment, the condition is resistant hypertension. In one embodiment, the administration of the RXFP1 modulator and the SGLT2 inhibitor is separate, sequential, or simultaneous.

In one embodiment, is provided a method of reducing blood pressure in a human patient in need of such treatment, comprising administering to the human patient a therapeutically effective amount of an RXFP1 modulator, wherein the RXFP1 modulator is administered in combination with a therapeutically effective amount of an SGLT2 inhibitor, and wherein the RXFP1 modulator is selected from: Compound 1, Compound 2, Compound 3, Compound 4, Compound 5 and Compound 6, or a pharmaceutically acceptable salt thereof. In one embodiment, the RXFP1 modulator is Compound 1 or a pharmaceutically acceptable salt thereof. In one embodiment, the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof. In one embodiment, the SGLT2 inhibitor is dapagliflozin. In one embodiment, the administration of the RXFP1 is modulator and the SGLT2 inhibitor is separate, sequential, or simultaneous. In one embodiment, the systolic blood pressure of the human patient is reduced. In one embodiment, the diastolic blood pressure of the human patient is reduced. In one embodiment, the systolic and diastolic blood pressures of the human patient are reduced.

In one embodiment, is provided a method of reducing blood pressure in a human patient in need of such treatment, comprising administering to the human patient a first amount of an RXFP1 modulator, and a second amount of an SGLT2 inhibitor, where the first amount and the second amount together comprise a therapeutically effective amount, and wherein the RXFP1 modulator is selected from: Compound 1, Compound 2, Compound 3, Compound 4, Compound 5 and Compound 6, or a pharmaceutically acceptable salt thereof. In one embodiment, the RXFP1 modulator is Compound 1 or a pharmaceutically acceptable salt thereof. In one embodiment, the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof. In one embodiment, the SGLT2 inhibitor is dapagliflozin. In one embodiment, the administration of the RXFP1 modulator and the SGLT2 inhibitor is separate, sequential, or simultaneous. In one embodiment, the systolic blood pressure of the human patient is reduced. In one embodiment, the diastolic blood pressure of the human patient is reduced. In one embodiment, the systolic and diastolic blood pressures of the human patient are reduced.

In one embodiment, there is provided a method as disclosed in any of the embodiments herein wherein the RXFP1 modulator is a compound as claimed or exemplified in International Patent Application No. PCT/EP2021/084673 or U.S. patent application Ser. No. 17/457,953 (both applications are incorporated by reference in their entirety).

As used herein, the term “heart failure” includes acute heart failure, chronic heart failure (CHF) and acute decompensated heart failure (ADHF). The term “heart failure” may also include more specific diagnoses such as heart failure with preserved ejection fraction (HFpEF), heart failure with mid-range ejection fraction (HFmrEF; also referred to as heart failure with mildly reduced ejection fraction), or heart failure with reduced ejection fraction (HFrEF). This may also include heart failure due to hypertrophic cardiomyopathy or dilated cardiomyopathy.

As used herein, the term “pulmonary hypertension” may be defined as a subject with a mean Pulmonary Arterial Pressure of about 20 mmHg or greater, optionally 25 mmHg or greater, typically when the subject is at rest. It may also be defined as a mean Pulmonary Arterial is Pressure of about 30 mmHg or greater, typically when the subject is or has recently been exercising. Thus, the subject may have a mean Pulmonary Arterial Pressure in the range of about 20 mmHg to about 30 mmHg, optionally about 25 mmHg to about 30 mmHg, or greater.

Alternatively or additionally, the subject may have:
a. a Right Ventricular Systolic Pressure of about 40 mmHg or greater; and/or
b. a Pulmonary Vascular Resistance of:
   i. less than 3.0 wood units; or
   ii. 3.0 or more wood units.

Thus, in some cases, the pulmonary hypertension may be classified as Group 2 pulmonary hypertension, as defined by the World Health Organisation. In other cases, the pulmonary hypertension may be classified as Group 1 pulmonary arterial hypertension, as defined by the World Health Organisation (see Ryan et al., 2012, Pulm. Circ. 2(1):107-121).

Parameters of pulmonary hypertension and heart failure may be measured or estimated using techniques known in the art. For instance, these include echocardiography, pulmonary artery catheter and implantable monitoring device.In certain embodiments, the subject may have been fitted with a blood pressure monitoring device, optionally a pulmonary artery pressure monitoring device, as are known in the art. In particular embodiments, the pulmonary artery pressure monitoring device is a CardioMEMS pressure monitoring device. Typically, the device is fitted prior to treatment with an RXFP1 modulator as described herein. Alternatively, the subject is fitted with the device during or after the period of treatment.

As used herein, the term “heart failure with pulmonary hypertension” refers to the subset of heart failure subjects who simultaneously suffer from pulmonary hypertension (HF+PH subjects).

As used herein, the term “resistant hypertension” is defined as the blood pressure of a hypertensive patient that remains elevated above goal despite the concurrent use of optimized doses of 3 antihypertensive agents of different classes, one of which is a diuretic, or a patient whose blood pressure is adequately controlled when receiving 4 or more antihypertensive medications concurrently (Carey et al., Hypertension, 2018, 72, e53-e90). The initial treatment of hypertension may be a calcium channel blocker (CCB), a blocker of the renin-angiotensin system (angiotensin-converting enzyme [ACE] inhibitor or angiotensin receptor blocker [ARB]), and a diuretic. For patients with rHT, further treatment may include a mineralocorticoid-receptor antagonist (MRA), a beta-blocker, and/or or a alpha-blocker. The subject with resistant hypertension may have a systolic blood pressure ≥140 mm Hg and/or diastolic blood pressure ≥90 mm Hg, typically when the subject is at rest. Alternatively, the subject with resistant is hypertension may have a systolic blood pressure ≥130 mm Hg and/or diastolic blood pressure ≥80 mm Hg, typically when the subject is at rest. Alternatively, the subject with resistant hypertension may have a systolic blood pressure ≥150 mm Hg and/or diastolic blood pressure ≥90 mm Hg, typically when the subject is at rest. In some instances, the resistant hypertension may be resistant essential hypertension. Essential hypertension, also known as primary hypertension, is a form of hypertension with no known secondary cause identified.

### Pharmaceutical Compositions

The combinations of RXFP1 modulators and SGLT2 inhibitors described herein may be administered as pharmaceutical compositions, comprising one or more pharmaceutically acceptable excipients.

Therefore, in one embodiment there is provided a pharmaceutical composition comprising an RXFP1 modulator, an SGLT2 inhibitor, and a pharmaceutically acceptable excipient, wherein the RXFP1 modulator is selected from: Compound 1, Compound 2, Compound 3, Compound 4, Compound 5 and Compound 6, or a pharmaceutically acceptable salt thereof.

In one embodiment, the RXFP1 modulator is Compound 1 or a pharmaceutically acceptable salt thereof. In one embodiment, the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof. In one embodiment, the SGLT2 inhibitor is dapagliflozin.

In one embodiment, there is provided a method as disclosed in any of the embodiments herein wherein the RXFP1 modulator is administered as a pharmaceutical composition comprising the RXFP1 modulator and a pharmaceutically acceptable excipient.

In one embodiment, there is provided a pharmaceutical composition comprising an RXFP1 modulator, an SGLT2 inhibitor and a pharmaceutically acceptable excipient for use in a method as described herein, wherein the RXFP1 modulator is selected from: Compound 1, Compound 2, Compound 3, Compound 4, Compound 5 and Compound 6, or a pharmaceutically acceptable salt thereof.

In one embodiment, there is provided a pharmaceutical composition comprising an RXFP1 modulator, an SGLT2 inhibitor, and a pharmaceutically acceptable excipient, wherein the RXFP1 modulator is a compound as claimed or exemplified in International Patent Application No. PCT/EP2021/084673 or U.S. patent application Ser. No. 17/457,953 (both applications are incorporated by reference in their entirety). In one embodiment, the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof. In one embodiment, the SGLT2 inhibitor is dapagliflozin.

The excipient(s) selected for inclusion in a particular composition will depend on factors such as the mode of administration and the form of the composition provided. Suitable pharmaceutically acceptable excipients are well known to persons skilled in the art and are described, for example, in the Handbook of Pharmaceutical Excipients, Sixth edition, Pharmaceutical Press, edited by Rowe, Ray C; Sheskey, Paul J; Quinn, Marian. Pharmaceutically acceptable excipients may function as, for example, adjuvants, diluents, carriers, stabilisers, flavourings, colorants, fillers, binders, disintegrants, lubricants, glidants, thickening agents and coating agents. As persons skilled in the art will appreciate, certain pharmaceutically acceptable excipients may serve more than one function and may serve alternative functions depending on how much of the excipient is present in the composition and what other excipients are present in the composition.

The pharmaceutical compositions may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing), or as a suppository for rectal dosing. The compositions may be obtained by conventional procedures well known in the art. Compositions intended for oral use may contain additional components, for example, one or more colouring, sweetening, flavouring and/or preservative agents.

### Kits

In one embodiment, there is provided a kit comprising:
a first pharmaceutical composition comprising an RXFP1 modulator selected from: Compound 1, Compound 2, Compound 3, Compound 4, Compound 5 and Compound 6, or a pharmaceutically acceptable salt thereof, and a pharmaceutically acceptable excipient; and
a second pharmaceutical composition comprising an SGLT2 inhibitor and a pharmaceutically acceptable excipient.

In one embodiment, the RXFP1 modulator is Compound 1 or a pharmaceutically acceptable salt thereof. In one embodiment, the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof. In one embodiment, the SGLT2 inhibitor is dapagliflozin.

### EXAMPLES

The compounds described in this specification are further illustrated in the following Examples. These Examples are given by way of illustration only and are non-limiting.

In the examples, high resolution mass spectra were recorded on a Micromass LCT mass spectrometer equipped with an electrospray interface (LC-HRMS).

¹H NMR measurements were performed on Bruker Avance III 300, 400, 500 and 600 spectrometers, operating at ¹H frequencies of 300, 400, 500 and 600 MHz, respectively. The experiments were typically recorded at 25° C. Chemical shifts are given in ppm with the solvent as internal standard. Protons on heteroatoms such as NH and OH protons are only reported when detected in NMR and can therefore be missing. The following abbreviations have been used (and derivatives thereof, e.g. dd, doublet of doublets, etc.): s, singlet; d, doublet; t, triplet; q, quartet; m, multiplet; br, broad; qn, quintet; p, pentet.

Flash chromatography was performed using either normal phase silica FLASH+® (40M, 25M or 12M), Biotage® SNAP Cartridges KP-Sil (340, 100, 50 or 10), or Agela® Flash Column Silica-CS Cartridges (330, 180, 120, 80) unless otherwise stated.

Reversed phase flash chromatography was performed using Agela® C-18 spherical 20-35 μm 100A cartridges unless otherwise stated.

In general, all solvents used were commercially available and of analytical grade. Anhydrous solvents were routinely used for reactions.

Phase Separators used in the examples are ISOLUTE® Phase Separator columns.

The Intermediates and Examples named below were named using ChemDraw Professional version 19.0.0.22 from PerkinElmer.

The following abbreviations were used
Aq Aq
B₂Pin₂ 4,4,5,5-Tetramethyl-2-(4,4,5,5-tetramethyl-1,3,2-dioxaborolan-2-yl)-1,3,2-dioxaborolane
Calcd Calculated
DCM Dichloromethane
DIA Diisopropylamine
DIAD Diisopropyl (E)-diazene-1,2-dicarboxylate
DIPEA N-Ethyl-N-isopropyl-propan-2-amine
DMF N,N-dimethylformamide
DMSO Dimethylsulfoxide
DPPA Diphenylphosphoryl azide
DTBBPY 4,4′-Di-tert-butyl-2,2′-dipyridyl
EDC 3-(Ethyliminomethyleneamino)-N,N-dimethyl-propan-1-amine;hydrochloride
ESI Electrospray ionization
Et Ethyl
Et₂O Diethyl ether
EtOAc Ethylacetate
EtOH Ethanol
h/hr Hour(s)
HATU (Dimethylamino)-N,N-dimethyl(3-oxido-1H-[1,2,3]triazolo[4,5-b]pyridinyl)methaniminium hexafluorophosphate
HOBt 1-Hydroxybenzotriazole;hydrate
HPLC High performance liquid chromatography
HRMS High resolution mass spectrometry
IPA Isopropyl alcohol
IPAC Isopropyl acetate
[Ir(COD)OMe]₂ Bis(1,5-cyclooctadiene)di-μ-methoxydiiridium(I)
L Litre
Me Methyl
MeCN Acetonitrile
mL Millilitre
MeOH Methanol
2-Me-THF 2-Methyltetrahydrofuran
Min Minutes
MS Mass spectrometry
MTBE Methyl tert-butyl ether
NMR Nuclear magnetic resonance
OAc Acetate
PE Petroleum ether
Pd/C Palladium on charcoal
Rt Room temperature
Sat Saturated
SFC Supercritical fluid chromatography
T3P 2,4,6-tripropyl-1,3,5,2,4,6-trioxatriphosphinane 2,4,6-trioxide
TEA Triethylamine
TFA Trifluoroacetic acid
THF Tetrahydrofuran
TLC Thin layer chromatography

### INTERMEDIATES

### Intermediate 1: Ethyl 8-methyl-1,4-dioxaspiro[4.5]decane-8-carboxylate

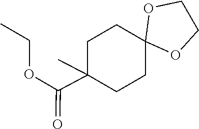 [SMILES: CCOC(=O)C1(C)CCC2(CC1)OCCO2]

A solution of DIA (576 mL, 413 g, 4.08 mol) in THF (3.50 L) was cooled to −50 to −40° C. and a solution of n-BuLi (2.5 M in hexane, 1.09 kg, 3.92 mol) was added over 3 h, maintaining the temperature between −50 to −40° C. The solution was stirred for 3 h at −50 to −40° C., followed by the addition of a solution of ethyl 1,4-dioxaspiro[4.5]decane-8-carboxylate (700 g, 3.27 mol, 2.34 M in THF) over 2 h, maintaining the temperature between −50 to −40° C. The reaction mixture was stirred for 4 h at −50 to −40° C. before the addition of methyl iodide (603 g, 4.25 mol, 3.04 M in THF) over 3 h, maintaining the temperature between −50 to −30° C. The reaction mixture was further stirred for 2 h at −50 to −30° C. followed by the addition of aq NH₄Cl (3.50 L, 20% w/w in H₂O) over 1 h, maintaining the temperature <0° C. The solution was warmed to between 15 to 25° C., held for 0.5 h then the layers were separated and the organic layer washed with aq NH₄Cl (2×3.50 L, 20% w/w in H₂O). Exchange of the organic reaction solvent from THF to EtOH under reduced pressure, maintaining the temperature <45° C., gave the title compound as a 27% w/w solution in EtOH (2.51 kg, 2.91 mol, 89%). ¹H NMR for purified compound (400 MHz CDCl₃) δ 1.18 (3H, s), 1.27-1.22 (3H, m), 1.47-1.35 (2H, m), 1.70-1.56 (4H, m), 2.13 (2H, d), 3.92 (4H, s), 4.14 (2H, q), MS (ESI): m/z [M+H]⁺ 229.2.

### Intermediate 2: 8-Methyl-1,4-dioxaspiro[4.5]decane-8-carboxylic acid

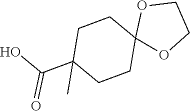

To a solution of Intermediate 1 (1.13 kg, 1.31 mol, 27% w/w in EtOH) was added EtOH (900 mL) followed by aq NaOH (2.63 L, 5.26 mol, 2 M in H₂O) maintaining the temperature between 15 to 30° C. The solution was heated to between 50 to 60° C., then held for 6 h before cooling to 15 to 30° C. and concentration of the solution to between 1.8 to 2.4 L under reduced pressure. Hexane (1.50 L) was added and the layers separated. The aq layer was collected and the pH adjusted to between 3 to 4 by the addition of aq HCl (1.30 L, 5.2 mol, 4 M in H₂O) maintaining the temperature <20° C. This aq solution was extracted with DCM (2×1.50 L) and the combined organic phases were concentrated under reduced pressure, maintaining the temperature <30° C. to give the title compound as a 17% w/w solution in DCM (1.43 kg, 1.24 mol, 94%). ¹H NMR for purified compound (500 MHz CDCl₃) δ 1.25 (3H, s), 1.53 (2H, dt), 1.62-1.72 (4H, m), 2.05-2.19 (2H, m), 3.93 (4H, s). MS (ESI): m/z [M+Na]⁺223.1.

### Intermediate 3: 1-Methyl-4-oxocyclohexane-1-carboxylic acid

### Method A

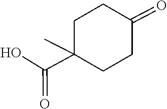

To a solution of Intermediate 2 (367 g, 250 mmol, 14% w/w in DCM) was added TFA (95.3 mL, 142 g, 1.25 mol). The reaction temperature was maintained between 25 to 35° C. for 20 h before cooling to between 0 to 10° C. H₂O (250 mL) was added to the reaction solution and the pH of the aq phase adjusted to between 9 and 10 by the addition of aq NaOH (440 mL, 1.76 mol, 4 M in H₂O). The layers were separated and the aq layer was retained and cooled to between 0 to 10° C. The pH was adjusted to between 2 and 3 by the addition of aq HCl (73.5 mL, 294 mmol, 4 M in H₂O) then extracted with DCM (3×250 mL) and the combined DCM solutions concentrated to between 150 to 200 mL under reduced pressure. Exchange of the organic reaction solvent from DCM to MeCN under reduced pressure, maintaining the temperature <40° C., gave the title compound as a 30% w/w solution in MeCN (119 g, 227 mmol, 91%). ¹H NMR for purified compound (400 MHz CDCl3) δ 1.39 (3H, s), 1.73 (2H, td), 2.43 (6H, m). MS (ESI): m/z [M+H]⁺ 157.1.

### Method B

To a solution of Intermediate 2 (6.17 kg, 3.83 mol, 12.4% in DCM) was added TFA (1.42 L, 2.18 kg, 19.13 mol). The reaction temperature was maintained between 25 to 35° C. for 20 h before cooling to between 0 to 10° C. A solution of aq NaOH (918 g, 22.96 mol dissolved in 7.66 L H₂O) was added to the reaction solution and the pH of the aqueous phase was adjusted to between 9 and 11. The layers were separated and the aq layer was retained and cooled to between 0 to 10° C. Addition of DCM (3.83 L) followed by aq HCl (1.52 L, 6.08 mol, 4 M in H₂O) adjusts the pH to between 3 and 4. The organic layer was retained and the aqueous extracted with DCM (2×3.83 L) and the combined organic phase was washed with brine (2.3 L, 15% w/w NaCl). The organic phase was concentrated under reduced pressure to 2.3 to 3.1 L. Exchange of the organic reaction solvent from DCM to MeCN under reduced pressure, maintaining the temperature <45° C., gave the title compound as a 18% solution in MeCN (2.85 kg, 3.32 mol, 87%). ¹H NMR for purified compound (400 MHz CDCl3) δ 1.39 (3H, s), 1.73 (2H, td), 2.43 (6H, m). MS (ESI): m/z [M+H]⁺ 157.1.

### Intermediate 4: Naphthalen-1-ylmethyl 1-methyl-4-oxocyclohexane-1-carboxylate

### Method A

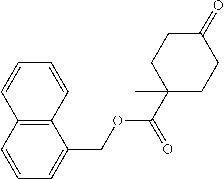 [SMILES: CC1(C(=O)OCc2cccc3ccccc23)CCC(=O)CC1]

To a solution of Intermediate 3 (119 g, 192 mmol, 25% w/w in MeCN) was added 1-chloromethylnaphthalene (32.2 g, 183 mmol) followed by DIPEA (70.0 mL, 49.7 g, 384 mmol) and NaI (2.88 g, 19.2 mmol). The solution was heated to between 50 to 60° C. for 8 h before cooling to between 0 to 10° C. H₂O (240 mL) was added and the pH of the reaction mixture adjusted to between 3 and 4 by the addition of aq HCl (55.0 mL, 220 mmol, 4 M in H₂O). The reaction mixture was extracted with MTBE (2×150 mL) and the combined organic phases washed with aq NaHCO₃ (150 mL, 144 mmol, 8% w/w in H₂O). The organic reaction solvent was exchanged from MTBE to IPA under reduced pressure, maintaining the temperature <40° C. The temperature of the reaction solution was lowered to between −10 to 3° C. and the solution stirred for 2 h, upon which a solid precipitate formed. The solids were filtered and dried under N₂ for 15 h to give the title compound as a white solid (42.8 g, 144 mmol, 74%); ¹H NMR (500 MHz, CDCl₃) 1.30 (3H, s), 1.65 (2H, td), 2.16-2.47 (6H, m), 5.66 (2H, s), 7.46 (1H, dd), 7.51-7.63 (3H, m), 7.78-7.93 (2H, m), 7.93-8.05 (1H, m). MS (ESI): m/z [M+Na]⁺319.1.

### Method B

To a solution of Intermediate 3 (2.66 kg, 3.09 mol, 18.2% in MeCN) was added 1-chloromethylnapthalene (535 g, 2.94 mol) followed by potassium carbonate (513 g, 3.71 mol) and a further portion of fresh MeCN (714 mL). The suspension was heated to between 50 to 60° C. for 17 h before cooling to 25 to 30° C. The solid was removed by filtration through a Celite pad, which was washed through with MeCN (2×967 mL). Concentrate the filtrates to 1.45 to 1.93 L under reduced pressure. The MeCN was exchanged to isopropanol under reduced pressure, maintaining the temperature <50° C. The temperature of the mixture was lowered to between 20 to 25° C., upon which a solid precipitate formed. The mixture was cooled further to −10 to 0° C., and the solids were then filtered, washed with isopropanol and dried under N₂ to give the title compound as a white solid (752.6 g, 2.49 mol, 80.5%); ¹H NMR (500 MHz, CDCl₃) 1.30 (3H, s), 1.65 (2H, td), 2.16-2.47 (6H, m), 5.66 (2H, s), 7.46 (1H, dd), 7.51-7.63 (3H, m), 7.78-7.93 (2H, m), 7.93-8.05 (1H, m). MS (ESI): m/z [M+Na]⁺319.1.

### Intermediate 5: Methyl 5-(1,3,6,2-dioxazaborocan-2-yl)-4-fluoro-2-methoxybenzoate Method A

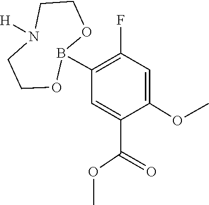 [SMILES: COC(=O)c1cc(B2OCCNCCO2)c(F)cc1OC]

B₂Pin₂ (362 g, 1.43 mol) was added to 2-Me-THF (1.75 L) that had been degassed with N₂ to <1% oxygen. The solution was held between 20 to 30° C. and methyl 4-fluoro-2-methoxybenzoate was added (250 g, 1.36 mol). DTBBPY (1.09 g, 4.10 mmol) was added and the reaction vessel evacuated and re-filled with N₂ until the oxygen level was <0.5%. [Ir(COD)OMe]₂ (1.35 g, 2.04 mmol) was added and the reaction vessel evacuated and re-filled with N₂ until the oxygen level was <0.5%. The reaction mixture was heated to between 80 to 85° C. and held at that temperature for a further 2 h. The reaction mixture was cooled to between 0 to 5° C. followed by the slow addition of diethanolamine (428 g, 4.07 mol, 10.9 M in IPA) over a period of 2.5 h, with the concurrent generation of H₂ gas. The reaction mixture was stirred for 2.5 h between 0 to 5° C., followed by filtration and washing of the solids with 2-Me-THF (3×750 mL). The solid was dried under N₂ for 10 h to give the title compound as a white solid (356 g, 1.20 mol, 88%); ¹H NMR (500 MHz, DMSO-d6) δ 2.81-2.89 (2H, m), 3.14 (2H, dq), 3.71 (2H, ddd), 3.74 (3H, s), 3.78 (3H, s), 3.84 (2H, td), 6.77 (1H, d), 7.10 (1H, s), 7.83 (1H, d). MS is (ESI): m/z [M+H]⁺ 297.1.

### Method B

B₂Pin₂ (29.0 g, 114 mmol) and methyl 4-fluoro-2-methoxybenzoate (20.6 g, 109 mmol) were added to 2-Me-THF (140 mL) that had been degassed with N₂ to <1% oxygen. The solution was held between 20 to 30° C. then DTBBPY (88 mg, 0.33 mmol) and [Ir(COD)OMe]₂ (108 mg, 0.16 mmol) were added and the reaction vessel evacuated and re-filled with N₂ until the oxygen level was <0.5%. The reaction mixture was heated to between 80 to 85° C. and held at that temperature for a further 3 h. The reaction mixture was cooled to between 0 to 10° C. followed by the slow addition of isopropanol (12.4 mL, 218 mmol), with the concurrent generation of H₂ gas. Addition of seed (100 mg of Intermediate 5) followed by addition of diethanolamine (22.84 g, 218 mmol) dissolved in IPA (20 mL) gave a mobile slurry. The slurry was warmed to 20 to 30° C. and the solid collect by filtration. It was then washed with 2-Me-THF (160 ml) and the solid was dried under N₂ for 10 h to give the title compound as a white solid (29.1 g, 96 mol, 88%); ¹H NMR (500 MHz, DMSO-d6) δ 2.81-2.89 (2H, m), 3.14 (2H, dq), 3.71 (2H, ddd), 3.74 (3H, s), 3.78 (3H, s), 3.84 (2H, td), 6.77 (1H, d), 7.10 (1H, s), 7.83 (1H, d). MS (ESI): m/z [M+H]⁺ 297.1.

### Intermediate 6: Methyl 4-fluoro-5-hydroxy-2-methoxybenzoate

### Method A

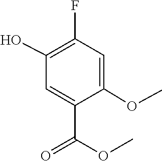

To a suspension of Intermediate 5 (350 g, 1.18 mol) in H₂O (1.05 L) was added THF (1.75 L) and the reaction mixture stirred until a clear solution is obtained. (NH₄)₂CO₃ (136 g, 1.41 mol) was added and the heterogenous mixture cooled to between 0 to 10° C. NaBO₃·4H₂O (217 g, 1.41 mol) was added in 10 equal portions over a period of 2 h maintaining the reaction temperature between 0 to 30° C. The reaction temperature was adjusted to between 20 to 30° C. and held for 1 h. An aq solution of NaHSO₃ (1.96 L, 942 mmol, 0.48 M in H₂O) was added over 3 h and the reaction mixture stirred for an additional 0.5 h. The reaction mixture was filtered, the solids washed with ethylacetate (700 mL) and the filtrate and wash combined to give a biphasic solution. The solution was separated and the retained organic phase solvent exchanged from THF/ethylacetate to MeOH under reduced pressure, maintaining the temperature <40° C. H₂O is (3.50 L) was added drop-wise over a period of 4 h and the reaction mixture cooled to between 0 to 5° C. and held for 2 h. The reaction mixture was filtered, the collected solids washed with H₂O (3×350 mL) and dried under hot air at <40° C. to give the title compound as a white solid (195 g, 974 mmol, 83% yield); ¹H NMR (500 MHz, CDCl₃) δ 3.82 (3H, s), 3.86 (3H, s), 6.72 (1H, d), 7.54 (1H, d). MS (ESI): m/z [M+H]⁺ 201.0.

### Method B

Intermediate 5 (32.41 g, 67.3 mmol) was dissolved in 2-Me-THF (100 mL) with acetic acid (12.13 g, 202 mmol) and cooled to between 0 to 10° C. Hydrogen peroxide solution (30% w/w, 9.16 g, 80.8 mmol) was added over 2 hours and then the reaction temperature was adjusted to between 20 to 30° C. and held for 18 hours. An aq solution of Na₂S₂O₃·5H₂O (20% w/w, 50 mL) quenches the mixture and gives a phase separation. The aqueous is discarded, and the organic washed twice with aq solution of Na₂S₂O₃·5H₂O (5% w/w, 100 mL). The organic phase was concentrated to 60 mL under reduced pressure followed by another 2 vacuum distillations with 2-Me-THF (100 mL) to give a dissolved solution at 35 to 45° C. Nucleation was controlled by addition of seed (100 mg of Intermediate 6) followed by slow addition of 300 mL n-heptane over 5 hours. The resulting slurry was adjusted to between 20 to 30° C. and stirred overnight prior to filtration. The collected solid was washed with n-heptane (2×60 mL) and dried to give the title compound as a white solid (12.5 g, 62.5 mmol, 93% yield); ¹H NMR (500 MHz, CDCl₃) δ 3.82 (3H, s), 3.86 (3H, s), 6.72 (1H, d), 7.54 (1H, d). MS (ESI): m/z [M+H]⁺ 201.0.

### Intermediate 7: (1R,2R,3S,4S)-3-(Methoxycarbonyl)bicyclo[2.2.1]hept-5-ene-2-carboxylic acid

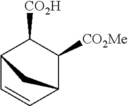 [SMILES: COC(=O)[C@@H]1[C@H](C(=O)O)[C@H]2C=C[C@@H]1C2]

To a solution of (3aR,4R,7S,7aS)-3a,4,7,7a-tetrahydro-4,7-methanoisobenzofuran-1,3-dione (387 g, 2.36 mol) in toluene (4.64 L) was added quinidine (843 g, 2.60 mol) followed by toluene (774 mL). The reaction mixture was cooled to between −10 to −5° C. and MeOH (227 g, 286 mL, 7.08 mol) was added drop-wise over 1.5 h before holding at between −10 to −5° C. for 14 h. The reaction mixture was warmed to between −5 to 5° C., held for 2 h, then filtered. The solids were washed with toluene (3×387 mL), the filtrate and washes combined and cooled to between 0 to 10° C. In a separate vessel an aq solution of HCl (590 mL, 7.08 mol, 12 M in H₂O) and NaCl (1.24 kg, 21.2 mol) were added to H₂O (6.39 L) and the resulting solution added dropwise to the is main reaction vessel, maintaining the reaction solution <10° C. The reaction mixture was warmed to between 10 to 20° C., held for 0.5 h then filtered. The solids were washed with toluene (1.94 L), the filtrate and wash combined, and the biphasic solution separated. The organic phase was washed with aq NaCl (3.87 L, 20% w/w in H₂O) and stored at <5° C. to give the title compound as a 5.9% w/w solution in toluene (6.19 kg, 1.83 mol, 78%); ¹H NMR for purified compound (400 MHz DMSO-d₆) δ 1.25-1.32 (1H, m), 1.95 (1H, d), 2.48-2.50 (2H, m), 2.93 (2H, s), 3.51 (3H, s), 6.15-6.22 (2H, m), 12.21 (1H, s). MS (ESI): m/z [M+Na]⁺219.1.

### Intermediate 8: Methyl (1S,2S,3R,4R)-3-aminobicyclo[2.2.1]hept-5-ene-2-carboxylate hydrochloride

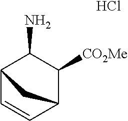 [SMILES: Cl ; COC(=O)[C@@H]1[C@H](N)[C@H]2C=C[C@@H]1C2]

To a solution of Intermediate 7 in toluene (6.19 kg, 5.9% w/w, 1.85 mol) at between −5 to 5° C. was added TEA (307 mL, 223 g, 2.22 mol) followed by DPPA (538 g, 1.94 mol), maintaining the reaction solution <5° C. The reaction mixture was stirred for 4 h at between −5 to 5° C. then TEA was added (767 mL, 557 g, 5.55 mol) followed by citric acid (352 g, 1.85 mol). The reaction mixture was stirred for 6 h at between −5 to 5° C. then H₂O (3.6 L) was added maintaining the reaction solution <10° C. The biphasic reaction solution was stirred for 0.5 h, the phases separated and the organic phase washed with H₂O (3.6 L) and aq NaCl (3.6 L, 15% w/w in H₂O) then stored between 2 to 8° C. to give methyl (1S,2S,3R,4R)-3-(azidocarbonyl)bicyclo[2.2.1]hept-5-ene-2-carboxylate (Intermediate 9) as a solution in toluene that was used directly in the next step. Intermediate 9 as a solution in toluene at between 2 to 8° C. was added over 2 h to a reactor containing toluene (1.80 L) at between 70 to 80° C., maintaining the reaction temperature <80° C. The resulting solution was stirred for 1 h before cooling to between 20 to 30° C. Exchange of the organic reaction solvent from toluene to 1,4-dioxane under reduced pressure, maintaining the temperature <50° C., gave Methyl (1S,2S,3R,4R)-3-isocyanatobicyclo[2.2.1]hept-5-ene-2-carboxylate (Intermediate 10) as a solution in 1,4-dioxane that was used directly in the next step. To a solution of Intermediate 10 in 1,4-dioxane at between 10 to 20° C. was added HCl (420 mL, 1.68 mol, 4 M in 1,4-dioxane) followed by H₂O (360 mL, 1.68 mol, 4.67 M in 1,4-dioxane). The reaction mixture was warmed to between 25 to 35° C. and held for 16 h. MTBE (1.65 L) was added drop-wise and the reaction mixture filtered, the solids washed with MTBE/1,4-dioxane (1:1, 660 mL) and MTBE (660 mL), is then dried at between 30 to 40° C. under vacuum to give the title compound as a white solid (258 g, 1.27 mol, >99% ee, 75%); ¹H NMR (400 MHz, DMSO-d6) δ 1.45 (1H, d), 2.04 (1H, d), 2.52-2.67 (1H, m), 2.94-3.10 (2H, m), 3.19 (1H, d), 3.65 (3H, s), 6.21 (1H, m), 6.30 (1H, m), 8.34 (3H, s). MS (ESI): m/z [M+H]⁺ 168.1.

### Intermediate 11: Naphthalen-1-ylmethyl (1r,4r)-4-hydroxy-1-methylcyclohexane-1-carboxylate

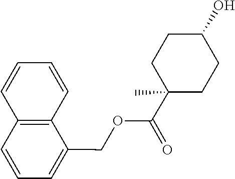 [SMILES: C[C@]1(C(=O)OCc2cccc3ccccc23)CC[C@H](O)CC1]

### Route A

To a solution of Na₂HPO₄·12H₂O (8.25 g, 23.0 mmol), NaH₂PO₄ (0.55 g, 4.48 mmol) and MgCl₂ (0.11 g, 1.10 mmol) in H₂O (550 mL) at 20 to 30° C. was added Intermediate 4 (50.0 g, 169 mmol) as a solution in IPA (450 mL). The pH of the reaction solution was adjusted to between 7.3 to 7.8 using 6 M HCl and NAD+(0.66 g, 1.00 mmol) was added followed by ADH-230 (7.50 g, 0.15 wt %). ADH-230 is an alcohol dehydrogenase available from Johnson Matthey PLC, UK (catalogue no. ADH-230). The reaction mixture was then held at 33 to 37° C. for 18 h before concentration to between 300 and 400 mL under reduced pressure, maintaining the temperature <45° C. NaCl (150 g), Celite® (20.0 g, 0.4 wt %) and MTBE (500 mL) was added and the reaction held for 0.5 h. The mixture was filtered and the filter cake washed with MTBE (250 mL). The combined filtrate was separated and the aq phase extracted with MTBE (500 mL). The organic phases were combined and washed with H₂O (250 mL) before solvent exchange to THF under reduced pressure, maintaining the temperature <45° C., gave the title compound (138 g, 33% w/w %, ≥99:1 trans:cis, <0.1% IPA, 92% yield) as a solution in THF that was used directly in the next step. ¹H NMR for purified compound (500 MHz, CDCl₃) δ 1.21 (3H, s), 1.48-1.58 (2H, m), 1.62-1.77 (4H, m), 1.82-1.93 (2H, m), 3.74-3.77 (1H, m), 5.57 (2H, s), 7.41-7.48 (1H, m), 7.48-7.57 (3H, m), 7.85 (1H, d), 7.87-7.91 (1H, m), 7.98 (1H, d). MS (ESI): m/z [M+Na]⁺321.1.

### Route B

A solution of lithium tri-sec-butylborohydride (1.06 g, 5.6 mmol) in THF (5 mL) was added dropwise to a stirred solution of Intermediate 4 (1.00 g, 3.37 mmol) in THF (10 mL) cooled to −78° C., over a period of 1 min under nitrogen. The resulting solution was stirred at −78° C. for 2 h. The reaction mixture was quenched with 0.1 M HCl (10 mL) at −78° C. and then extracted with EtOAc (3×50 mL). The organic layers were pooled and dried over Na₂SO₄ filtered and evaporated. The residue was purified by preparative TLC (EtOAc/PE, 1:3), to afford the title compound (0.488 g, 48.5%) as a pale yellow gum. The isolated material had a 3:100 cis/trans ratio. ¹H NMR (400 MHz, CDCl₃) δ1.21-1.25 (s, 3H), 1.37-1.49 (m, 1H), 1.49-1.61 (m, 2H), 1.61-1.74 (m, 4H), 1.83-1.95 (m, 2H), 3.74-3.83 (dq, 1H), 5.57-5.61 (s, 2H), 7.43-7.54 (dd, 1H), 7.50-7.61 (m, 3H), 7.84-7.94 (m, 2H), 7.97-8.04 (m, 1H).). MS (ESI): m z [M+Na]⁺321.

### Intermediate 12: Methyl 4-fluoro-2-methoxy-5-(((1s,4s)-4-methyl-4-((naphthalen-1-ylmethoxy)carbonyl)cyclohexyl)oxy)benzoate

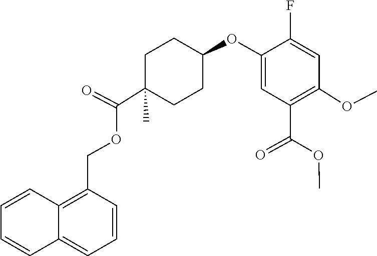 [SMILES: COC(=O)c1cc(O[C@H]2CC[C@@](C)(C(=O)OCc3cccc4ccccc34)CC2)c(F)cc1OC]

To a solution of Intermediate 11 in THF (736 g, 34% w/w, 839 mmol) was added THF (156 mL), PPh₃ (248 g, 944 mmol) and Intermediate 6 (140 g, 699 mmol). The solution was heated to 30° C. prior to the drop-wise addition of DIAD (184 g, 909 mmol) over 1 h maintaining the reaction temperature <40° C. The solution was held at between 3° and 40° C. for 1 h before cooling to between 2° and 30° C. followed by the addition of an aq solution of NaCl (700 mL, 20% w/w in H₂O). The layers were separated and the crude solution of the title compound in THF was used directly in the next step. ¹H NMR for purified compound (500 MHz, CDCl₃) δ 1.17 (3H, s), 1.20-1.30 (2H, m), 1.58 (2H, qd), 1.88-1.98 (2H, m), 2.29 (2H, d), 3.84 (3H, s), 3.88 (3H, s), 4.05 (1H, tq), 5.61 (2H, s), 6.72 (1H, d), 7.43-7.58 (5H, m), 7.82-7.94 (2H, m), 8.00 (1H, d). MS (ESI): m/z [M+Na]⁺503.2.

### Intermediate 13: 4-Fluoro-2-methoxy-5-(((1s,4s)-4-methyl-4-((naphthalen-1-ylmethoxy)carbonyl)cyclohexyl)oxy)benzoic acid

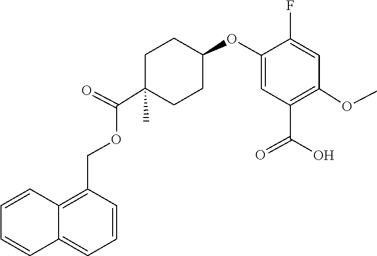 [SMILES: COc1cc(F)c(O[C@H]2CC[C@@](C)(C(=O)OCc3cccc4ccccc34)CC2)cc1C(=O)O]

To the crude solution of Intermediate 12 used directly from the previous step at between 0 and 5° C., was added a aq solution of LiOH·2H₂O (88.0 g, 2.10 mol, in 525 mL of H₂O) over 1 h maintaining the reaction temperature <10° C. The solution was warmed to between 15 and 30° C. and vigorously stirred for 16 h. IPAC (1.68 L) was added and the solution cooled to between 0 and 10° C. followed by the drop-wise addition of H₃PO₄ (1.26 L, 2.52 M, 2 M in H₂O), maintaining the reaction temperature <10° C., to give a solution pH of between 4.0 and 5.0. The organic layer was separated and washed with of an aq solution of NaCl (700 mL, 20% w/w in H₂O). The THF was removed under reduced pressure, maintaining the temperature <50° C. and IPAC (4.20 L) was added to give the title compound in IPAC that was used directly in the next step. ¹H NMR for purified compound (500 MHz, CDCl₃) δ 1.18 (3H, s), 1.22-1.36 (2H, m), 1.58 (2H, qd), 1.95 (2H, dt), 2.29 (2H, d), 4.02 (3H, s), 4.19 (1H, td), 5.60 (2H, s), 6.82 (1H, d), 7.46 (1H, dd), 7.49-7.62 (3H, m), 7.78 (1H, d), 7.82-7.94 (2H, m), 7.99 (1H, d).

### Intermediate 14: 4-Fluoro-2-methoxy-5-(((1s,4s)-4-methyl-4-((naphthalen-1-ylmethoxy)carbonyl)cyclohexyl)oxy)benzoate cyclohexanaminium salt

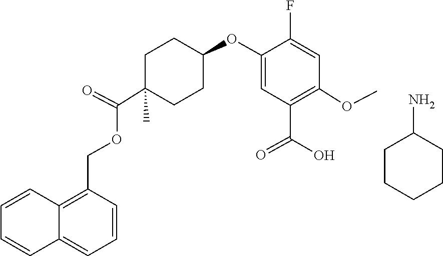

To a crude solution of Intermediate 13 in IPAC used directly from the previous step at between 50 and 55° C., was added a solution of cyclohexylamine (280 mL, 699 mmol, 2.5 M in IPAC) drop-wise over 3 h. The heterogenous slurry was stirred at between 5° and 55° C. for 0.5 h then at between 4° and 45° C. for a further 1 h. The reaction mixture was filtered and the solids washed with IPAC (3×0.98 L) pre warmed to between 4° and 45° C. and dried under a flow of N₂ at 45° C. for 16 h. To the dried collected solids was added MeOH (3.64 L) and the mixture heated to between 55 and 56° C. H₂O (1.58 L) was added drop-wise over 1 h then the mixture stirred for 1 h before cooling to between 0 and 5° C. over 3 h. The heterogenous slurry was held for a further 1 h then filtered, washed with 5:3 MeOH:H₂O at 0° C. (2×750 mL) and the solids dried under N₂ at 45° C. for 16 h to give the title compound as a white solid (332 g, 85% from methyl 4-fluoro-5-hydroxy-2-methoxybenzoate); ¹H NMR (500 MHz, CDCl₃) δ 0.96 (1H, ddt), 1.03-1.36 (6H, m), overlapping 1.14 (3H, S), 1.46-1.7 (5H, m), 1.91 (4H, dt), 2.26 (2H, d), 2.81 (1H, t), 3.76 (3H, s), 4.03 (1H, tt), 5.59 (2H, s), 6.65 (1H, d), 7.37-7.49 (2H, m), 7.49-7.6 (3H, m), 7.81-7.93 (2H, m), 7.98 (1H, d). MS (ESI): m/z [M+Na]⁺489.2.

### Intermediate 15: Methyl (1S,2S,3R,4R)-3-(4-fluoro-2-methoxy-5-(((1s,4S)-4-methyl-4-((naphthalen-1-ylmethoxy)carbonyl)cyclohexyl)oxy)benzamido)bicyclo[2.2.1]hept-5-ene-2-carboxylate

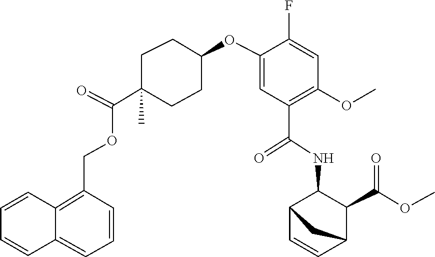 [SMILES: COC(=O)[C@@H]1[C@H](NC(=O)c2cc(O[C@H]3CC[C@@](C)(C(=O)OCc4cccc5ccccc45)CC3)c(F)cc2OC)[C@H]2C=C[C@@H]1C2]

To a solution of Intermediate 14 (149 g, 264 mmol) in DCM (750 mL) at between 15 and 30° C. was added H₂O (450 mL) followed by the slow addition of HCL (300 mL, 1 M in H₂O). The biphasic solution was stirred for 0.5 h then separated and the organic phased washed with HCl (750 mL, 0.2 M in H₂O) then with H₂O (3×750 mL). The organic solution was concentrated under reduced pressure, maintaining the temperature below 30° C., to dry to <0.1% H₂O. The solution was diluted with DCM (450 mL) to bring the total volume to 750 mL before the addition of Intermediate 8 (59.3 g, 291 mmol) to give a heterogenous slurry. To this mixture was added DIPEA (137 g, 1.06 mol) followed by T3P (252 g, 397 mmol, 50% w/w in EtAOc) and the solution stirred for 1 h. The solution was cooled to between 0 and 10° C. followed by the addition of H₂O (750 mL) and subsequently stirred for a further 0.5 h. The biphasic solution was separated and the organic phase washed with H₂O (2×750 mL) before solvent exchange to THF under reduced pressure gave the title compound in THF that was used directly in the next step. ¹H NMR for purified compound (500 MHz, CDCl₃) δ1.16 (3H, s), 1.25 (2H, td), 1.49-1.69 (3H, m), 1.92-2.01 (2H, m), 2.04-2.1 (1H, m), 2.28 (2H, d), 2.71 (1H, dd), 2.83 (1H, s), 2.92-3.05 (1H, m), 3.61 (3H, s), 3.93 (3H, s), 4.17 (1H, td), 4.46 (1H, td), 5.60 (2H, s), 6.25 (2H, ddd), 6.72 (1H, d), 7.46 (1H, dd), 7.48-7.6 (3H, m), 7.81-7.95 (3H, m), 7.99 (1H, d), 8.60 (1H, d). MS (ESI): m/z [M+H]⁺ 616.3.

### Intermediate 16: (1S,2S,3R,4R)-3-(4-Fluoro-2-methoxy-5-(((1s,4S)-4-methyl-4-((naphthalen-1-ylmethoxy)carbonyl)cyclohexyl)oxy)benzamido)bicyclo[2.2.1]hept-5-ene-2-carboxylic acid

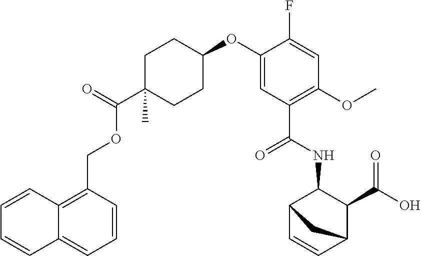 [SMILES: COc1cc(F)c(O[C@H]2CC[C@@](C)(C(=O)OCc3cccc4ccccc34)CC2)cc1C(=O)N[C@H]1[C@@H](C(=O)O)[C@@H]2C=C[C@H]1C2]

A crude solution of Intermediate 15 in THF (750 mL) from the previous step was cooled to between 0 and 5° C. An aq solution of LiOH·2H₂O (27.7 g, 661 mmol, in 150 mL of H₂O) was added and the solution held for 36 h. The pH of the solution was adjusted to 2 with the portion wise slow addition of HCl (0.5 M, 1.45 L, 2.90 mol) and held for 1 h between 0 and 5° C. The heterogenous slurry was filtered and the solids washed with 1:3 MeOH:H₂O at 0° C. (600 mL) and the solids dried under N₂ at 45° C. for 16 h to give crude title compound as a white solid (158 g, 99%). The crude (150 g) was slurried in IPAC (1.13 L) at between 6° and 65° C. for 0.5 h. The heterogenous mixture was cooled to between 0 and 5° C. over 3 h then further stirred for 1 h before filtration. The collected solids were with IPAC at between 0 and 5° C. (2×300 mL) then dried under N₂ at 45° C. for 12 h to give the title compound as a white solid (127 g, 82% from Intermediate 14); ¹H NMR (500 MHz, CDCl₃) δ1.16 (3H, s), 1.2-1.35 (2H, m), 1.50-1.69 (3H, m), 1.89-2.08 (3H, m), 2.27 (2H, ddd), 2.72 (1H, dd), 2.80 (1H, s), 3.06 (1H, s), 3.75 (3H, s), 4.15 (1H, tt), 4.43-4.54 (1H, m), 5.59 (2H, s), 6.24 (2H, ddd), 6.53 (1H, d), 7.45 (1H, dd), 7.47-7.58 (3H, m), 7.8-7.9 (3H, m), 7.94-8.05 (1H, m), 8.59 (1H, d). MS (ESI): m/z [M+H]⁺ 602.3.

### Intermediate 17: Naphthalen-1-ylmethyl (1S,4s)-4-(2-fluoro-4-methoxy-5-(((1R,2R,3S,4S)-3-(((1-methylcyclobutyl)methyl)carbamoyl)bicyclo[2.2.1]hept-5-en-2-yl)carbamoyl)phenoxy)-1-methylcyclohexane-1-carboxylate

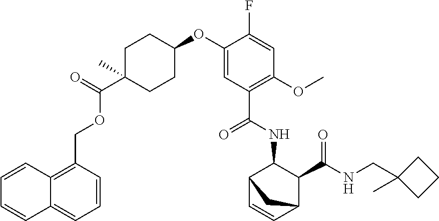 [SMILES: COc1cc(F)c(O[C@H]2CC[C@@](C)(C(=O)OCc3cccc4ccccc34)CC2)cc1C(=O)N[C@H]1[C@@H](C(=O)NCC2(C)CCC2)[C@@H]2C=C[C@H]1C2]

To a solution of DIPEA (6.45 g, 49.9 mmol) in DCM (300 mL) at between 0 and 5° C. was added Intermediate 16 (30.6 g, 49.9 mmol) followed by (1-methylcyclobutyl)methanamine hydrochloride (8.63 g, 62.4 mmol). DIPEA (25.8 g, 200 mmol) was added drop-wise maintaining the temperature between 0 and 5° C., followed by the addition of T3P (50.8 g, 79.8 mmol, 50% w/w in EtAOc) over 0.5 h. The solution was warmed to between 15 and 25° C. and stirred for 1 h followed by the drop-wise addition of H₂O (150 mL) maintaining the temperature below 30° C. The biphasic solution was separated and the organic phase washed with H₂O (2×150 mL) then the solvent exchanged to EtOH under reduced pressure to give the title compound as a crude solution in EtOH (128 g, 26% w/w, 96% yield) that was used directly in the next step.

¹H NMR for purified compound (500 MHz, CDCl₃) δ 0.98 (3H, s), 1.16 (3H, s), 1.21-1.29 (2H, m), 1.51-1.66 (5H, m), 1.66-1.76 (3H, m), 1.76-1.82 (1H, m), 1.88-2.02 (2H, m), 2.26 (3H, dd), 2.40 (1H, dd), 2.80 (1H, s), 3.00 (1H, s), 3.05 (1H, dd), 3.21 (1H, dd), 3.93 (3H, s), 4.06-4.2 (1H, m), 4.39 (1H, td), 5.60 (2H, s), 5.64 (1H, t), 6.19-6.38 (2H, m), 6.70 (1H, d), 7.46 (1H, dd), 7.49-7.62 (3H, m), 7.75-7.93 (3H, m), 8.00 (1H, d), 8.66 (1H, d). MS (ESI): m/z [M+H]⁺ 683.3.

### Intermediate 19: (1R,2S,3R,4S)-3-(5-(((1s,4S)-4-Carboxy-4-methylcyclohexyl)oxy)-4-cyano-2-methoxybenzamido)bicyclo[2.2.1]heptane-2-carboxylic acid

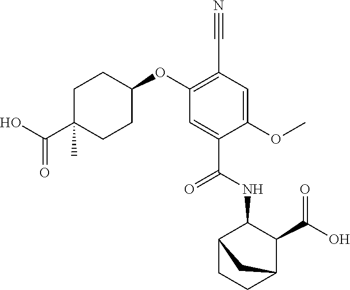 [SMILES: COc1cc(C#N)c(O[C@H]2CC[C@@](C)(C(=O)O)CC2)cc1C(=O)N[C@@H]1[C@H]2CC[C@H](C2)[C@@H]1C(=O)O]

### Step A. Intermediate 20: Methyl 4-cyano-2-methoxy-5-(((1s,4s)-4-methyl-4-((naphthalen-1-ylmethoxy)carbonyl)cyclohexyl)oxy)benzoate

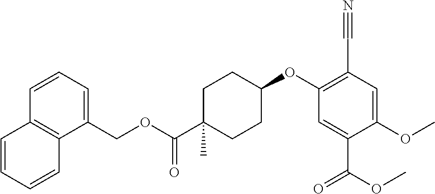 [SMILES: COC(=O)c1cc(O[C@H]2CC[C@@](C)(C(=O)OCc3cccc4ccccc34)CC2)c(C#N)cc1OC]

A solution of methyl 4-cyano-5-hydroxy-2-methoxybenzoate (1.4 g, 6.7 mmol), Intermediate 11 and PPh₃ (2.6 g, 10.1 mmol) in THF (30 mL) was stirred at 60° C. for 10 min. After slow addition of DIAD (1.97 mL, 10.1 mmol), the reaction mixture was stirred at 60° C. for 14 h. The solvent was then removed under reduced pressure and the residue redissolved in EtOAc (150 mL), washed sequentially with NaHCO₃ (sat, 200 mL), NH₄Cl (sat, 250 mL) and brine (sat, 250 mL). The organic layer was separated and dried over Na₂SO₄, filtered and the solvent removed under reduced pressure. The crude product was purified by flash chromatography using a gradient of 0-18% EtOAc in PE as mobile phase to give the title compound (3.25 g, 99%) as a white solid. MS (ESI): m/z [M+Na]⁺510.3.

### Step B. Intermediate 21: 4-Cyano-2-methoxy-5-(((1s,4s)-4-methyl-4-((naphthalen-1-ylmethoxy)carbonyl)cyclohexyl)oxy)benzoic acid

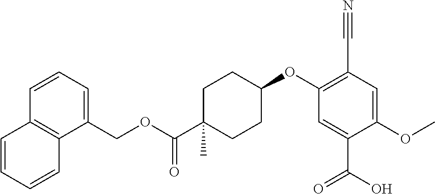 [SMILES: COc1cc(C#N)c(O[C@H]2CC[C@@](C)(C(=O)OCc3cccc4ccccc34)CC2)cc1C(=O)O]

LiOH (1.6 g, 66.7 mmol) was added portionwise to a stirred solution of Intermediate 20 (3.25 g, 6.7 mmol) in H₂O:THF 1:3 (80 mL) at 10° C. and the resulting suspension was stirred at 20° C. After 3 h, the pH of the reaction mixture was adjusted to pH 3 by the addition of HCl (2 M). The reaction mixture was diluted with EtOAc (350 mL), and washed sequentially with brine (sat, 350 mL), H₂O (350 mL), and brine (350 mL). The organic layer was separated, dried over Na₂SO₄, filtered and the solvent removed under reduced pressure. The crude product was purified by crystallisation from IPA/EtOAc to afford the title compound (3.16 g, 100%) as a white solid. MS (ESI): m/z [M+Na]⁺ 496.3.

### Step C. Intermediate 22: Methyl (1S,2S,3R,4R)-3-(4-cyano-2-methoxy-5-(((1s,4S)-4-methyl-4-((naphthalen-1-ylmethoxy)carbonyl)cyclohexyl)oxy)benzamido)bicyclo[2.2.1]hept-5-ene-2-carboxylate

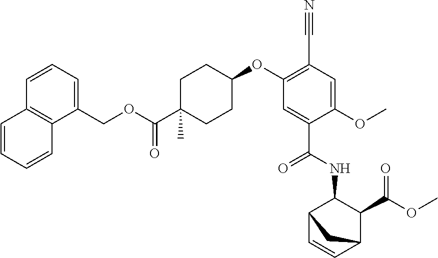 [SMILES: COC(=O)[C@@H]1[C@H](NC(=O)c2cc(O[C@H]3CC[C@@](C)(C(=O)OCc4cccc5ccccc45)CC3)c(C#N)cc2OC)[C@H]2C=C[C@@H]1C2]

DIPEA (3.5 mL, 20 mmol) was added to a solution of Intermediate 21 (3.16 g, 6.67 mmol), Intermediate 8 (1.291 g, 6.34 mmol), EDC (1.9 g, 10 mmol) and HOBt (1.533 g, 10.01 mmol) in DMF (60 mL) at 10° C. and the resulting suspension was stirred at rt for 13 hours. The reaction mixture was diluted with EtOAc (500 mL) and washed sequentially with NH₄Cl (sat, 200 mL), H₂O (300 mL), and brine (sat, 250 mL). The organic layer was dried over Na₂SO₄, filtered and the solvent removed under reduced pressure. The crude product was purified by flash chromatography using a gradient of 0-20% EtOAc in PE as mobile phase to afford the title compound (2.6 g, 62%) as a white solid. MS (ESI): m/z [M+H]⁺ 623.4.

### Step D. Intermediate 23: (1S,4s)-4-(2-Cyano-4-methoxy-5-(((1S,2R,3S,4R)-3-(methoxycarbonyl)bicyclo[2.2.1]heptan-2-yl)carbamoyl)phenoxy)-1-methylcyclohexane-1-carboxylic acid

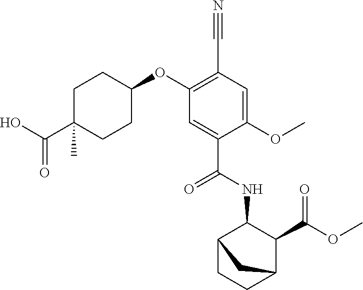 [SMILES: COC(=O)[C@H]1[C@@H]2CC[C@@H](C2)[C@H]1NC(=O)c1cc(O[C@H]2CC[C@@](C)(C(=O)O)CC2)c(C#N)cc1OC]

Intermediate 22 (5.7 g, 9.15 mmol) and Pd/C (0.584 g, 0.55 mmol) in MeOH (100 mL) was stirred at 20° C. under an atmosphere of hydrogen (1.5 atm) for 14 h. The mixture was filtered through a Celite® pad and the solvent was removed under reduced pressure. The crude product was purified by crystallisation from EtOAc/EtOH to afford the title compound (5.1 g) as a pale yellow solid. MS (ESI): m/z [M+H]⁺ 485.4.

### Step E. (1R,2S,3R,4S)-3-(5-(((1s,4S)-4-Carboxy-4-methylcyclohexyl)oxy)-4-cyano-2-methoxybenzamido)bicyclo[2.2.1]heptane-2-carboxylic acid

A solution of LiOH (50 mL, 52.6 mmol, 1.05 M in H₂O) was added to a stirred solution of Intermediate 23 (5.1 g, 10.5 mmol) in THF (100 mL) at 10° C. The reaction mixture was allowed to warm to rt and stirred for 14 h, then acidified to pH 2 using HCl (1 M, aq). The reaction mixture was diluted with EtOAc (350 mL), and washed sequentially with brine (300 mL, sat), H₂O (300 mL) and brine (300 mL, sat). The organic phase was separated and dried over Na₂SO₄, filtered and the solvent was removed under reduced pressure. The crude product was purified by precipitation from EtOAc/Et₂O followed by reversed phase flash chromatography on a C18 column using a gradient of 0-50% MeCN in HCl (0.4%, aq) as mobile phase to afford the title compound (4.00 g, 82%) as a white solid; ¹H NMR (400 MHz, DMSO-d6) δ 1.13 (s, 3H), 1.20 (s, 1H), 1.23 (s, 2H), 1.33 (t, 2H), 1.46 (q, 4H), 1.84 (d, 1H), 1.92 (d, 2H), 2.03-2.14 (m, 4H), 2.38 (d, 1H), 2.67 (d, 1H), 3.89 (s, 3H), 4.23 (t, 1H), 4.43 (dt, 1H), 7.54 (s, 1H), 7.59 (s, 1H), 8.67 (d, 1H), 12.30 (s, 2H). MS (ESI): m/z [M+H]⁺ 471.3.

### EXAMPLES

### Example 1: (1S,4s)-4-(2-Fluoro-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid (Form A)

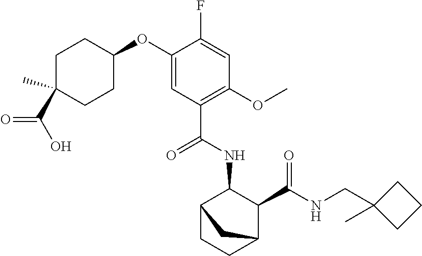 [SMILES: COc1cc(F)c(O[C@H]2CC[C@@](C)(C(=O)O)CC2)cc1C(=O)N[C@@H]1[C@H]2CC[C@H](C2)[C@@H]1C(=O)NCC1(C)CCC1]

To a solution of Intermediate 17 in EtOH (206 g 19% w/w, 57.9 mmol) was added EtOH (385 mL) followed by 10 wt % Pd/C (3.96 g, 5% w/w). The vessel was purged with N₂ six times followed by H₂ a further six times. The vessel was pressurized to 0.4 MPa with H₂ and the reaction solution stirred for 20 h at between 2° and 30° C. The H₂ atmosphere was completely replaced with N₂ before the reaction mixture was filtered and the solids washed with EtOH (3×80 mL). A second identical batch was conducted and the collected EtOH solutions combined to give a single solution. The solvent was exchanged to EtOAc under reduced pressure maintaining the temperature below 45° C. The EtOAc solution (280 mL) was heated to between 7° and 75° C. for 0.5 h then cooled to between 4° and 45° C. and n-heptane (475 mL) added drop-wise over 0.5 h. The mixture was stirred for 0.5 h then cooled to between 2° and 25° C. over 2 h then held for a further 2 h. The heterogenous slurry was filtered then the solids washed twice with 1:2 EtOAc/n-heptane (160 mL) prior to drying at below 45° C. for 20 h to give crude title compound as a white solid (55.7 g, 87%).

Part 1: The crude title compound (2.50 g, 4.59 mmol) was dissolved in EtOH (15.0 mL). The temperature of the solution was maintained at 25.0±2.0° C. during the drop-wise addition of water (7.50 mL) during which a precipitate formed. The heterogenous slurry was stirred for a further 1.0 h then collected via filtration. The solids were washed with a (2:3) mixture of EtOH/Water (2×5.00 mL), collected and dried under N₂ to give the title compound as a white is solid (1.80 g, 72%). This material was characterized as Form A and used as seed following the method described in Part 2.

Part 2: The crude title compound (50.0 g, 91.8 mmol) was dissolved in EtOH (350 mL) then passed through a filter. EtOH (100 mL) was added to vessel then passed through the filter to give a combined EtOH solution. The temperature of the solution was maintained at 25.0±2.0° C. during the slow addition of H₂O (150 mL) over 0.5 h. The solution was stirred for a further 0.5 h then seed material from Part 1 (0.005 g, 0.1% w/w) was added. The solution was held for 6 h then cooled to 20.0±0.5° C. over 2 h, then held for a further 6 h. H₂O (150 mL) was added slowly over 6 h then the mixture held for a further 2 h prior to filtration. EtOH (45 mL) and H₂O (30 mL) was added to vessel then used to wash the filter cake. The solids were collected and dried under N₂ at below 45° C. for 12 h to give the title compound Form A as a white solid (42.2 g, 85%); ¹H NMR (500 MHz, CDCl₃) δ 0.97 (3H, s), 1·12-1.42 (5H, m), overlapping 1.25 (3H, S), 1.43-1.82 (10H, m), 1.92-2.1 (3H, m), 2.25 (3H, dd), 2.51 (2H, dd), 2.96 (1H, dd), 3.18 (1H, dd), 3.92 (3H, s), 4·12-4.28 (1H, m), 4.41 (1H, t), 5.81 (1H, t), 6.70 (1H, d), 7.86 (1H, d), 8.60 (1H, d). HRMS (ESI) m/z [M+H]⁺ calcd for C₃₀H₄₂FN₂O₆: 545.3022 found: 545.3019.

### Example 2: (1S,4s)-4-(5-(((1S,2R,3S,4R)-3-((cyclobutylmethyl)carbamoyl)bicyclo[2.2.1]heptan-2-yl)carbamoyl)-2-fluoro-4-methoxyphenoxy)-1-methylcyclohexane-1-carboxylic acid

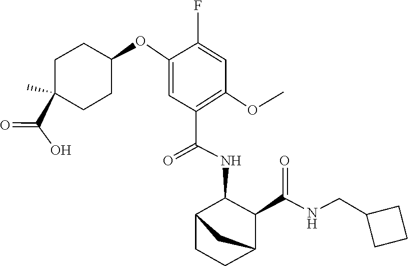 [SMILES: COc1cc(F)c(O[C@H]2CC[C@@](C)(C(=O)O)CC2)cc1C(=O)N[C@@H]1[C@H]2CC[C@H](C2)[C@@H]1C(=O)NCC1CCC1]

### Step A Intermediate 18: Naphthalen-1-ylmethyl (1S,4s)-4-(5-(((1R,2R,3S,4S)-3-((cyclobutylmethyl)carbamoyl)bicyclo[2.2.1]hept-5-en-2-yl)carbamoyl)-2-fluoro-4-methoxyphenoxy)-1-methylcyclohexane-1-carboxylate

To a solution of DIPEA (1.07 g, 8.31 mmol) in DCM (50.5 mL) at between 0 and 5° C. was added Intermediate 16 (5.00 g, 8.31 mmol) followed by cyclobutylmethanamine hydrochloride (1.26 g, 10.4 mmol). DIPEA (4.21 g, 32.6 mmol) was added drop-wise maintaining the temperature between 0 and 5° C., followed by the addition of T3P (8.46 g, 13.3 mmol, 50% w/w in EtOAc) over 0.5 h. The solution was warmed to between 15 and 25° C. and stirred for 2 h followed by the drop-wise addition of water (25.0 mL) maintaining the temperature below 30° C. The biphasic solution was separated and the organic phase washed with water (2×25.0 mL) then the solvent exchanged to EtOH (75.0 mL) under reduced pressure to give the title compound as a solution in EtOH that was used directly in the next step.

### Step B: (1S,4s)-4-(5-(((1S,2R,3S,4R)-3-((cyclobutylmethyl)carbamoyl)bicyclo[2.2.1]heptan-2-yl)carbamoyl)-2-fluoro-4-methoxyphenoxy)-1-methylcyclohexane-1-carboxylic acid

To the EtOH solution of Intermediate 18 was added 10 wt % Pd/C (290 mg, 5% w/w). The vessel was purged with N₂ six times followed by H₂ a further six times. The vessel was pressurized to 0.4 MPa with H₂ and the reaction solution stirred for 20 h at between 2° and 30° C. The H₂ atmosphere was completely replaced with N₂ before the reaction mixture was filtered and the solids washed with EtOH (2×12.2 ml). The solvent was exchanged to EtAOc under reduced pressure maintaining the temperature below 45° C. The EtAOc solution (41.0 mL) was heated to between 7° and 75° C. for 0.5 h then cooled to between 4° and 45° C. and n-heptane (34.8 mL) added drop-wise over 0.5 h. The mixture was stirred for 0.5 h then cooled to between 20 and 25° C. over 2 h then held for a further 2 h. The heterogenous slurry was filtered then the solids washed twice with 1:2 EtOAc/n-heptane (11.6 mL) prior to drying at below 45° C. for 20 h to give the title compound as a white solid (3.28 g, 74%). ¹H NMR (500 MHz, DMSO-d₆) δ 1.04-1.30 (6H, m), overlapping 1.10 (3H, s), 1.35-1.58 (5H, m), 1.59-1.69 (2H, m), 1.71-1.82 (2H, m), 1.83-1.92 (2H, m), 1.95-2.02 (1H, m), 2.01-2.10 (3H, d), 2.18-2.31 (2H, m), 2.50-2.55 (1H, d), 2.92-3.00 (1H, m), 3.06-3.14 (1H, m), 3.89 (3H, s), 4.07-4.17 (2H, m), 7.11 (1H, d), 7.67 (1H, d), 7.94 (1H, t), 8.83 (1H, d). MS (ESI): m/z [M+H]⁺ 531.3.

### Example 3. (1S,4s)-4-(2-Cyano-5-(((1S,2R,3S,4R)-3-((cyclopropylmethyl)carbamoyl)bicyclo[2.2.1]heptan-2-yl)carbamoyl)-4-methoxyphenoxy)-1-methylcyclohexane-1-carboxylic acid

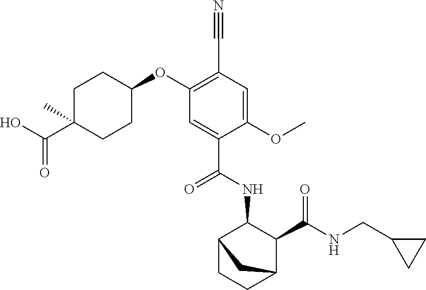 [SMILES: COc1cc(C#N)c(O[C@H]2CC[C@@](C)(C(=O)O)CC2)cc1C(=O)N[C@@H]1[C@H]2CC[C@H](C2)[C@@H]1C(=O)NCC1CC1]

A solution of Intermediate 19 (500 L, 0.024 g, 0.05 mmol, 0.1 M in DMF), a solution of cyclopropylmethanamine (500 L, 0.05 mmol, 0.1 M in DMF) and a solution of DIPEA (500 L, 0.15 mmol, 0.3 M in DMF) were added to a vial at rt. A solution of HATU (500 L, 0.15 mmol, 0.3 M in DMF) was added and the reaction mixture was stirred at 40° C. overnight. The crude mixture was washed with DMSO (3×500 L), filtered and the filtrate was evaporated under reduced pressure. The crude product was purified by preparative SFC on a Phenomenex Luna HILIC column (5 μm 250×30 ID mm) using MeOH/20 mM NH₃ in CO₂ as mobile phase to afford the title compound (10.3 mg, 39%). HRMS (ESI) m/z [M+H]⁺ calcd for C₂₉H₃₈N₃O₆: 524.2756 found: 524.2752.

### Example 4: (1S,4s)-4-(2-Cyano-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid

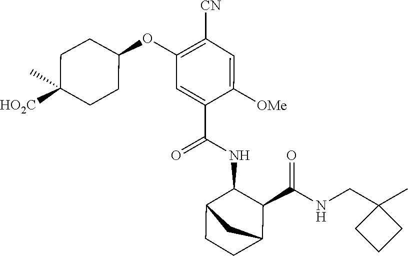

### Step A: Intermediate 24: (1S,2S,3R,4R)-3-(4-Cyano-2-methoxy-5-(((1s,4S)-4-methyl-4-((naphthalen-1-ylmethoxy)carbonyl)cyclohexyl)oxy)benzamido)bicyclo[2.2.1]hept-5-ene-2-carboxylic acid

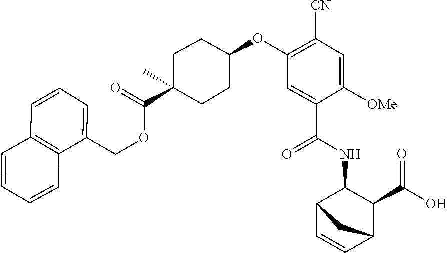 [SMILES: COc1cc(C#N)c(O[C@H]2CC[C@@](C)(C(=O)OCc3cccc4ccccc34)CC2)cc1C(=O)N[C@H]1[C@@H](C(=O)O)[C@@H]2C=C[C@H]1C2]

2 M aq LiOH (10.6 mL, 21.12 mmol) was added to a solution of Intermediate 22 (2.60 g, 4.18 mmol) in DME (50 mL), then the reaction mixture was stirred at rt for 12 hr. 10% aq citric acid was added to the reaction mixture until pH<3, then the reaction mixture was extracted with EtOAc twice and the combined organic layer was concentrated in vacuo to give titled compound (2.5 g, 97%). MS (ESI) m z 609.3 [M+H]⁺.

### Step B: Intermediate 25: Naphthalen-1-ylmethyl (1S,4s)-4-(5-(((1R,2R,3S,4S)-3-(((1-methylcyclobutyl)methyl)carbamoyl)bicyclo[2.2.1]hept-5-en-2-yl)carbamoyl)-2-cyano-4-methoxyphenoxy)-1-methylcyclohexane-1-carboxylate

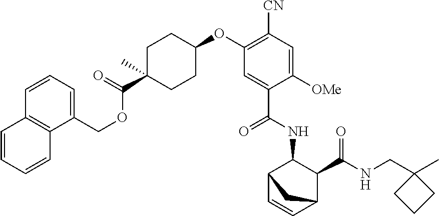 [SMILES: COc1cc(C#N)c(O[C@H]2CC[C@@](C)(C(=O)OCc3cccc4ccccc34)CC2)cc1C(=O)N[C@H]1[C@@H](C(=O)NCC2(C)CCC2)[C@@H]2C=C[C@H]1C2]

HATU (344 mg, 0.904 mmol) and DIPEA (0.43 mL, 2.46 mmol) were added to Intermediate 24 (500 mg, 0.821 mmol) and (1-methylcyclobutyl)methylamine hydrochloride (134 mg, 0.986 mmol) in DMF (4 mL), then the mixture was stirred at rt for 30 min. H₂O (50 mL) was added to the reaction mixture and the residual precipitate was collected by filtration, then the residue was dried under vacuum pump to give the title compound (611 mg, 100%). MS (ESI) m z 690.4 [M+H]⁺.

### Step C: (1S,4s)-4-(2-Cyano-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid

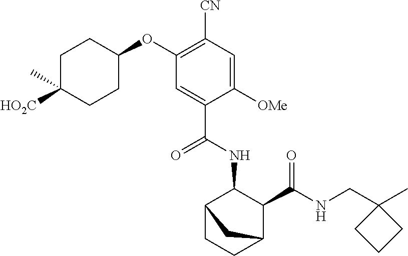

Palladium (10% Pd/C, moisture by 50% H₂O, 200 mg) was added to a solution of Intermediate 25 (609 mg, 0.883 mmol) in MeOH (4.4 mL). The reaction mixture was stirred under 1 atm of hydrogen atmosphere at rt for 3 hr. The hydrogen in the reaction vessel was replaced with argon and the reaction mixture was filtered with Celite®. After the filtrate was concentrated in vacuo, the crude product was purified by flash chromatography using a gradient of 0-5% MeOH in CHCl₃ as mobile phase to give titled compound (389 mg, 80%). ¹H NMR (400 MHz, DMSO-d6) δ 0.99 (s, 3H), 1.21 (s, 3H), 1.23-1.41 (m, 5H), 1.45-1.85 (m, 10H), 1.96-2.13 (m, 3H), 2.18-2.29 (m, 3H), 2.35-2.40 (m, 1H), 2.65-2.71 (m, 1H), 3.06 (dd, J=13.2, 5.8 Hz, 1H), 3.17 (dd, J=13.2, 6.3 Hz, 1H), 3.98 (s, 3H), 4.26-4.33 (m, 1H), 4.34-4.43 (m, 1H), 7.37 (s, 1H), 7.72 (s, 3H), 7.84-7.91 (m, 1H), 9.09 (d, J=8.5 Hz, 1H). HRMS (ESI) m/z [M+H]⁺ calcd for C₃₁H₄₂N₃O₆: 552.3068 found: 552.3064.

### Example 5: (1S,4s)-4-(2-cyano-4-methoxy-5-(((1S,2R,3S,4R)-3-(neopentylcarbamoyl)bicyclo[2.2.1]heptan-2-yl)carbamoyl)phenoxy)-1-methylcyclohexane-1-carboxylic acid

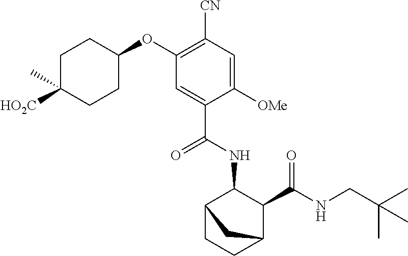 [SMILES: COc1cc(C#N)c(O[C@H]2CC[C@@](C)(C(=O)O)CC2)cc1C(=O)N[C@@H]1[C@H]2CC[C@H](C2)[C@@H]1C(=O)NCC(C)(C)C]

The titled compound was prepared analogously to Example 3, using 2,2-dimethylpropan-1-amine instead of cyclopropylmethanamine. HRMS (ESI) m/z [M+H]⁺ calcd for C₃₀H₄₂N₃O₆: 540.3068 found: 540.3076.

### Example 6: (1S,4s)-4-(2-cyano-5-(((1S,2R,3S,4R)-3-((3-fluorobicyclo[1.1.1]pentan-1-yl)carbamoyl)bicyclo[2.2.1]heptan-2-yl)carbamoyl)-4-methoxyphenoxy)-1-methylcyclohexane-1-carboxylic acid

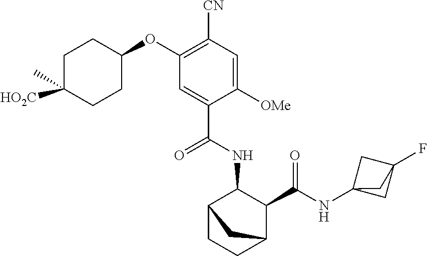 [SMILES: COc1cc(C#N)c(O[C@H]2CC[C@@](C)(C(=O)O)CC2)cc1C(=O)N[C@@H]1[C@H]2CC[C@H](C2)[C@@H]1C(=O)NC12CC(F)(C1)C2]

The titled compound was prepared analogously to Example 3, using 3-fluorobicyclo[1.1.1]pentan-1-amine instead of cyclopropylmethanamine. HRMS (ESI) m z [M+H]⁺ calcd for C₃₀H₃₇FN₃O₆: 554.2666 found: 554.2670.

### Biological and Physicochemical Data

RXFP1 Hu cAMP (Test A)

To screen for modulators of hRXFP1, an assay identifying compounds that stimulate cAMP production via the Gs-coupled hRXFP1 receptor was used. cAMP HiRange HTRF kit (available from CisBio Bioassays, France; catalogue number 62AM6PEJ) was employed in large according to manufacturer's recommendations for detection of cAMP. The HTRF method is a competitive immunoassay between native cAMP produced by cells and cAMP labeled with the dye d2. The tracer binding is visualized with a cryptate labeled antibody for cAMP and the signal is thus inversely proportional to the amount of produced cAMP.

### Preparation of Assay Reagents:

Assay buffer: HBSS (ThermoFisher, 14065) with 5 mM Hepes (ThermoFisher, 15630) pH 7.4 containing 0.1% BSA (Sigma, A8806)

Cells: Jump-In™ T-REx™ CHO-Kl Cells (ThermoFisher) stably transfected with human RXFP1 was employed. Cells were induced to express human RXFP1 by treatment with 10 ng/ml doxycycline for 24 h. Cells were then cryopreserved for long term storage. At the start of each experiment, cells were thawn, washed with PBS and resuspended in assay buffer to 1.875*10\hat{ }5 cells/ml

cAMP standard: stock standard cAMP provided in the CisBio kit was diluted in assay buffer to a top final concentration of 2.8 μM in the assay.

HTRF detection reagents: cAMP-d2 and anti-cAMP cryptate reconstituted according to CisBio instructions were diluted 1:40 in lysis buffer provided with the HTRF-kit.

### Step by Step Procedure for Running the Assay:

1. 40 nL test compounds dissolved in DMSO were aquostically dispensed (Labcyte Echo) to white 384-well plates (Greiner; 784075), sealed and stored at room temperature until assayed.

2. 40 nL 200 nM Relaxin-2 in DMSO (1 nM final concentration) was added to 100% control wells and 40 nL DMSO added to 0% wells with Echo acoustic dispenser at the day of assay.

3. 4 μL assay buffer with 1 mM IBMX (0.5 mM final concentration) to block phosphodiesterases was added with Multidrop Combi (ThermoFisher).

4. 4 μL cell solution at 1.875*10≡cells/ml was added with Multidrop Combi to give 750 cells/well.

5. 45 min incubation at room temperature.

6. 4 μL cAMP-d2 in lysis buffer was added with Multidrop Combi.

7. 4 μL anti-cAMP cryptate in lysis buffer was added with Multidrop Combi

8. 2 h incubation at room temperature

9. Homogenous Time-Resolved Fluorescence (HTRF) signal was detected with an Envision (PerkinElmer) or Pherastar (BMG Labtech) reader (λex=340 nm, λem=665 and 615 nm).

Using a cAMP standard curve, HTRF data was converted to amount cAMP produced in the samples which was subsequentially used for calculation of concentration responses. Concentration response data were fitted with a four parameter logistic fit, the Hill equation. The results from the assay are reported in Table 1 as EC₅₀ (μM) and S_{inf} (%).

EC₅₀ is defined as the concentration at which the stimulatory activity reaches 50% of its maximum level. Where the assay was run multiple times for the same compound, the geometric mean is reported.

S_{inf} is the fitted activity level, efficacy, at infinite concentration of test compound. To facilitate comparison of efficacy data, efficacy was normalized to % effect of the response stimulated by a saturating concentration of relaxin (1 nM). Where the assay was run multiple times for the same compound, the arithmetic mean is reported.

### Human Plasma Protein Binding (Test B)

The assay was conducted according to the Human Plasma Protein Binding Assay described in pages 167-170 of Wemevik, J. et al., “A Fully Integrated Assay Panel for Early Drug Metabolism and Pharmacokinetics Profiling”, Assay and Drug Development Technologies, 2020, 18(4), 157-179. Data are reported in Table 1 as fraction unbound (fᵤ) (% free). Where the assay was run multiple times for the same compound, the arithmetic mean is reported.

### Human Liver Microsomal Stability (Test C)

The assay was conducted according to the Human Liver Microsome Stability Assay described in pages 170-174 of Wemevik, J. et al., “A Fully Integrated Assay Panel for Early Drug Metabolism and Pharmacokinetics Profiling”, Assay and Drug Development Technologies, 2020, 18(4), 157-179. Data are reported in Table 1 as CLᵢₙₜ (μl/min/mg protein). Where the assay was run multiple times for the same compound, the arithmetic mean is reported.

### Human Hepatocyte Stability (Test D)

The metabolic stability of compounds in human hepatocytes was assessed using the following protocol:

1. Prepare 10 mM stock solutions of compound and control compounds in appropriate is solvent (DMSO). Place incubation medium (L-15 Medium) in a 37° C. water bath, and allow warming for at least 15 minutes prior to use.

2. Add 80 μL of acetonitrile to each well of the 96-well deep well plate (“Quenching plate”).

3. In a new 96-well plate, dilute the 10 mM test compounds and the control compounds to 100 μM by combining 198 μL of acetonitrile and 2 μL of 10 mM stock solution.

4. Remove a vial of cryopreserved (less than −150° C.) human hepatocytes (LiverPool™ 10-Donor Human hepatocytes obtained from Bioreclamation IVT (Product No. SO1205)) from storage, ensuring that vials remain at cryogenic temperatures until thawing process ensues. As quickly as possible, thaw the cells by placing the vial in a 37° C. water bath and gently shaking the vials. Vials should remain in water bath until all ice crystals have dissolved and are no longer visible. After thawing is complete, spray vial with 70% ethanol, transfer the vial to a bio-safety cabinet.

5. Open the vial and pour the contents into the 50 mL conical tube containing thawing medium. Place the 50 mL conical tube into a centrifuge and spin at 100 g for 10 minutes (room temperature). Upon completion of spin, aspirate thawing medium and resuspend hepatocytes in enough incubation medium to yield ˜1.5×10⁶ cells/mL.

6. Using Cellometer® Vision, count cells and determine the viable cell density. Cells with poor viability (<80% viability) are not acceptable for use. Dilute cells with incubation medium to a working cell density of 1.0×10⁶ viable cells/mL.

7. Transfer 247.5 μL of hepatocytes into each well of a 96-well cell incubation plate. Place the plate on Eppendorf Thermomixer Comfort plate shaker to allow the hepatocytes to warm for 10 minutes.

8. Add 2.5 μL of 100 μM test compound or control compounds into an incubation well containing cells to initiate the reaction.

9. Incubate the plate at 37° C. and 900 rpm on an Eppendorf Thermomixer Comfort plate shaker. At 0.5, 5, 15, 30, 45, 60, 80, 100 and 120 min, transfer 20 μL of the incubated mixture to a separate “Quenching plate”, then mix the sample by vortex for 2 min.

10. Centrifuge the quenching plates for 20 minutes at 4,000 rpm. Transfer 30 μL of supernatant of each compound into a 96-well analysis plate. 4 compounds are pooled together into one cassette. Then dilute the pooled sample by adding of 180 μl of pure water. All incubations are performed in singlicate.

All calculations were carried out using Microsoft Excel. Peak areas were determined from extracted ion chromatograms. In vitro intrinsic clearance (in vitro Clᵢₙₜ, in μL/min/10⁶ cells) of parent compound was determined by regression analysis of the Ln percent parent disappearance vs. time curve. The in vitro intrinsic clearance (in vitro Clᵢₙₜ, in μL/min/10⁶ cells) is reported in Table 1, and was determined from the slope value using the following equation:

$$in vitro {Cl}_{int} = kV / N$$

V=incubation volume (0.25 mL);
N=number of hepatocytes per well (0.25×10⁶ cells)

Where the assay was run multiple times for the same compound, the geometric mean is reported.

### Rat Hepatocyte Stability (Test E)

The assay was conducted according to the Rat Hepatocyte Stability Assay described in pages 170-174 of Wernevik, J. et al., “A Fully Integrated Assay Panel for Early Drug Metabolism and Pharmacokinetics Profiling”, Assay and Drug Development Technologies, 2020, 18(4), 157-179. Data are reported in Table 1 as mean Clᵢₙₜ (μl/min/10⁶ cells). Where the assay was run multiple times for the same compound, the geometric mean is reported.

### Solubility (Test F)

The assay was conducted according to the Solubility Assay described in pages 164-167 of Wemevik, J. et al., “A Fully Integrated Assay Panel for Early Drug Metabolism and Pharmacokinetics Profiling”, Assay and Drug Development Technologies, 2020, 18(4), 157-179. Data are reported in Table 1 as solubility (μM). Where the assay was run multiple times for the same compound, the arithmetic mean is reported.

TABLE 1
Assay data

| | | | | | Test D | Test E | |
|---|---|---|---|---|---|---|---|
| | | | Test B | Test C | Human | Rat | |
| | | | Human | Human Liver | Hepatocyte | Hepatocyte | |
| | | | Plasma | Microsomal | Stability | Stability | |
| | | | Protein | Stability | Clᵢₙₜ | Clᵢₙₜ | Test F |
| | Test A | | Binding | Clᵢₙₜ | (μL/min/10⁶ | (μL/min/10⁶ | Solubility |
| Example | EC₅₀ (μM) | S_{inf} (%) | (% free) | (μl/min/mg) | cells) | cells) | (μM) |
| 1 | 0.017 | 109 | 4.3 | 23 | 4.8 | 11 | >500 |
| 2 | 0.23 | 101 | 1.9 | 14 | 2 | 27 | >1000 |
| 3 | 0.39 | 97 | 17 | <3 | | 28 | 940 |
| 4 | 0.0058 | 100 | 6.3 | 19 | 5.6 | 18 | >890 |
| 5 | 0.024 | 114 | 12 | <3 | <1.2 | 15 | 910 |
| 6 | 0.024 | 105 | 17 | <5.1 | 1.7 | 11 | >860 |

Human RXFP1 cGMP Production Assay (Test G)

To profile compounds for RXFP1 agonist activity with respect to cGMP production, the Green GENIe cGMP Assay (Montana Molecular; catalogue number D800G) was employed. The assay is based on an mNeonGreen fusion protein fluorescent biosensor delivered to mammalian cells in a BacMam vector. Fluorescence is reduced when cGMP is bound to the biosensor.

### Preparation of Assay Reagents:

Assay buffer: DPBS (Gibco; 14040133) containing 0.1% BSA (Sigma; A8806)

Cells: HEK293s cells stably transfected with human RXFP1 in pIRESneo3 was is employed. Cells were cultured in DMEM medium (Gibco; 31966) with 10% FBS complemented with 0.8 mg/mL to maintain RXFP1 expression.

### Step by Step Protocol for Running the Assay:

### Day 1

1. Cells were splitted one day ahead of transduction and seeded to 63 000 cells/cm2 in DMEM medium with 10% FBS without antibiotics in a tissue culture flask.

### Day 2

2. After PBS wash cells were detached using accutase (Gibco; 1737341), resuspended in medium and collected in a 50 mL tube.
3. Cells were counted with a CEDEX (Innovatis) and diluted with medium to 267 000 cell/mL.
4. A viral transduction mastermix was prepared by mixing reagents in the following proportions for a single well:
   6 μL GENIe BacMAM vector
      0.2 μL 500 mM sodium butyrate
   13.8 μL DMEM medium with 10% FBS
   20 μL total volume
5. Cells and transduction mastermix were mixed in proportions 30 μL cells and 20 μL mastermix for a single well.
6. 50 μL cell-transduction mix from above was dispensed per well into Black poly-D-lysine coated μclear 384-well plates (Greiner; 781946).
7. Plate was incubated in the dark at 37° C., 5% CO₂ for 24 h.

### Day 3

8. Medium was removed from plates using a Bluewasher (BluCatBio).
9. 20 μL assay buffer was added with a Multidrop Combi (ThermoFisher).
10. Plate was incubated in the dark at room temperature for 30 min prior to assaying.
11. Plates were assayed using a FLIPR Tetra (Molecular Devices): 10 μL compound diluted with assay buffer was added to each well by the FLIPR Tetra and measuring green fluorescence over time for up to 3 h.

Data was processed using Screener software (Genedata AG). After subtraction of background fluorescence (before addition of compounds), area under curve values from 0 to 90 min after compound addition was used for calculation of responses. Concentration response data were fitted with a four parameter logistic fit and EC₅₀ values (nM) are reported in Table 2.

### Human RXFP1 Phospho-ERK Assay (Test H)

To profile compounds for RXFP1 agonist activity with respect to ERK phosphorylation, the advanced phospho-ERK (Thr202/Tyr204) cellular kit (CisBio; 64AERPEH) was employed. The assay uses two antibodies. One labeled with a donor fluorophore (Eu cryptate), a second with an acceptor (d2). The first antibody specific binds to phosphorylated ERK, the second binds another motif of ERK and independently of its phosphorylation state. ERK phosphorylation enables immune-complex formation involving the two antibodies, thereby generating a FRET signal. Its intensity is proportional to the concentration of phosphorylated ERK in the sample. Assay was performed according to manufacturers recommendations.

### Preparation of Assay Reagents:

Cells: HEK293s cells stably transfected with human RXFP1 in pIRESneo3 was employed. Cells were cultured in DMEM medium (Gibco; 31966) with 10% FBS complemented with 0.8 mg/mL to maintain RXFP1 expression. Assay was performed on cells kept in continuous culture.

Dilution of test compounds: Compounds were diluted to desired concentrations with serum-free DMEM without phenol red (Gibco; 31053-038). DMSO concentration was adjusted to 0.4%.

Antibody mix: The Eu and d2 labelled anti ERK1/2 antibodies were separately diluted 20-fold with detection buffer provided in the kit. Shortly prior to the experiment, equal volumes of each diluted antibody solution were combined to an antibody mix.

### Step by Step Protocol for Running the Assay:

### Day 1

1. Cells were detached from culture flasks using accutase (Gibco; 1737341), resuspended in DMEM medium without phenol red containing 10% FBS and collected in a 50 mL tube.
2. Cells were counted with a CEDEX (Innovatis) and diluted with the medium above to 320 000 cell/mL.
3. 100 μL cell suspension was dispensed per well into Black μclear poly-D-lysine coated μclear 96-well plates (Greiner; 655946).
4. Plates were incubated at 37° C., 5% CO₂ for 24 h.

### Day 2

5. Serum starvation: Medium was removed and replaced with 50 μL serum-free DMEM without phenol red. Plates were incubated at 37° C., 5% CO₂ for 5 h.
6. 50 μL test compound solutions were added per well.
7. Plates were incubated at room temperature for 5 min.
8. Stimulation was stopped by rapidly removing medium and adding 50 μL lysis buffer (diluted to 1× final concentration prior to addition) per well.
9. Plates were transferred to −80° C. and lysates frozen.

### Day 3

10. Plates were thawed and shaken at room temperature for 30 min.
11. Cells lysates were homogenized by pipetting.
12. 16 μL homogenate per well was transferred to white low volume 384-well plates (Greiner; 784075)
13. 4 μL antibody mix was added per well.
14. Plates were incubated at room temperature in the dark for 4 h.
15. Homogenous Time-Resolved Fluorescence (HTRF) signal was detected with a Pherastar (BMG Labtech) reader (λex=340 nm, λem=665 and 615 nm).

HTRF ratio data was processed using Screener software (Genedata AG). Concentration response data were fitted with a four parameter logistic fit and EC₅₀ value (nM) reported in Table 2.

TABLE 2
Assay Data

| | Test G | Test H |
|---|---|---|
| | cGMP | phospo-ERK |
| | assay EC₅₀ | assay EC₅₀ |
| Compound | (nM) | (nM) |
| Example 1 | 50 | 6.3 |
| Example 4 | 29 | 2.5 |
| Example 5 | 72 | 6.8 |
| Example 6 | 107 | 5.2 |
| relaxin2 | 0.085 | 0.038 |

Those skilled in the art will appreciate that the biological assays described above may be performed using alternative equipment and minor variations to the protocol without significantly is affecting the results.

This description and its specific examples, while indicating certain embodiments, are intended for purposes of illustration only. This disclosure, therefore, is not limited to the illustrative embodiments described in this specification, and may be variously modified. In addition, it is to be appreciated that various embodiments that are, for clarity reasons, described in the context of separate embodiments, also may be combined to form a single embodiment. Conversely, various embodiments that are, for brevity reasons, described in the context of a single embodiment, also may be combined to form sub-combinations thereof.

Any publications disclosed within the specification are hereby incorporated by reference.

1. An RXFP1 modulator for use in therapy of a human patient, wherein the RXFP1 modulator is administered in combination with an SGLT2 inhibitor, and wherein the RXFP1 modulator is selected from: 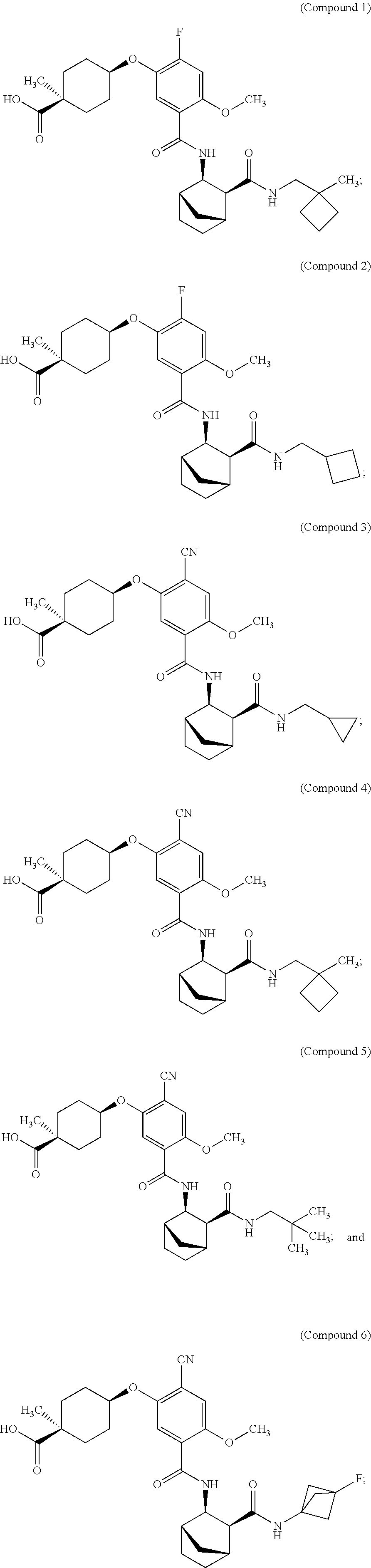
or a pharmaceutically acceptable salt thereof.

2. An RXFP1 modulator for use in the treatment of a condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient, wherein the RXFP1 modulator is administered in combination with an SGLT2 inhibitor, and wherein the RXFP1 modulator is selected from: Compound 1, Compound 2, Compound 3, Compound 4, Compound 5 and Compound 6, or a pharmaceutically acceptable salt thereof.

3. The RXFP1 modulator for use of claim 2, wherein the condition is hypertension.

4. The RXFP1 modulator for use of claim 2, wherein the condition is resistant hypertension.

5. The RXFP1 modulator for use of any one of the preceding claims, wherein the administration of the RXFP1 modulator and the SGLT2 inhibitor is separate, sequential, or simultaneous.

6. The RXFP1 modulator for use of any one of the preceding claims, wherein the RXFP1 modulator is Compound 1 or a pharmaceutically acceptable salt thereof.

7. The RXFP1 modulator for use of any one of the preceding claims, wherein the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof.

8. The RXFP1 modulator for use of any one of the preceding claims, wherein the SGLT2 inhibitor is dapagliflozin.

9. The RXFP1 modulator for use of any one of the preceding claims, wherein the RXFP1 modulator is Compound 1 or a pharmaceutically acceptable salt thereof, and the SGLT2 inhibitor is dapagliflozin.

10. A method of treating a condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient in need of such treatment, comprising administering to the human patient a therapeutically effective amount of an RXFP1 modulator, wherein the RXFP1 modulator is administered in combination with a therapeutically effective amount of an SGLT2 inhibitor, and wherein the RXFP1 modulator is selected from: Compound 1, Compound 2, Compound 3, Compound 4, Compound 5 and Compound 6, or a pharmaceutically acceptable salt thereof.

11. A method of treating a condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient in need of such treatment, comprising administering to the human patient a first amount of an RXFP1 modulator, and a second amount of an SGLT2 inhibitor, where the first amount and the second amount together comprise a therapeutically effective amount, and wherein the RXFP1 modulator is selected from: Compound 1, Compound 2, Compound 3, Compound 4, Compound 5 and Compound 6, or a pharmaceutically acceptable salt thereof.

12. The method of claim 10 or 11, wherein the condition is hypertension.

13. The method of claim 10 or 11, wherein the condition is resistant hypertension.

14. A method of reducing blood pressure in a human patient in need of such treatment, comprising administering to the human patient a therapeutically effective amount of an RXFP1 modulator, wherein the RXFP1 modulator is administered in combination with a therapeutically effective amount of an SGLT2 inhibitor, and wherein the RXFP1 modulator is selected from: Compound 1, Compound 2, Compound 3, Compound 4, Compound 5 and Compound 6, or a pharmaceutically acceptable salt thereof.

15. A method of reducing blood pressure in a human patient in need of such treatment, comprising administering to the human patient a first amount of an RXFP1 modulator, and a second amount of an SGLT2 inhibitor, where the first amount and the second amount together comprise a therapeutically effective amount, and wherein the RXFP1 modulator is selected from: Compound 1, Compound 2, Compound 3, Compound 4, Compound 5 and Compound 6, or a pharmaceutically acceptable salt thereof.

16. The method of any one of claims 10 to 15, wherein the administration of the RXFP1 modulator and the SGLT2 inhibitor is separate, sequential, or simultaneous.

17. The method of any one of claims 10 to 16, wherein the RXFP1 modulator is Compound 1 or a pharmaceutically acceptable salt thereof.

18. The method of any one of claims 10 to 17, wherein the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof.

19. The method of any one of claims 10 to 18, wherein the SGLT2 inhibitor is dapagliflozin.

20. The method of any one of claims 10 to 19, wherein the RXFP1 modulator is Compound 1 or a pharmaceutically acceptable salt thereof, and the SGLT2 inhibitor is dapagliflozin.

21. A pharmaceutical composition comprising an RXFP1 modulator, an SGLT2 inhibitor, and a pharmaceutically acceptable excipient, wherein the RXFP1 modulator is selected from: Compound 1, Compound 2, Compound 3, Compound 4, Compound 5 and Compound 6, or a pharmaceutically acceptable salt thereof.

22. The pharmaceutical composition of claim 21, wherein the RXFP1 modulator is Compound 1 or a pharmaceutically acceptable salt thereof.

23. The pharmaceutical composition of claim 21 or 22, wherein the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof.

24. The pharmaceutical composition of any one of claims 21 to 23, wherein the SGLT2 inhibitor is dapagliflozin.

25. The pharmaceutical composition of any one of claims 21 to 24, wherein the RXFP1 modulator is Compound 1 or a pharmaceutically acceptable salt thereof, and the SGLT2 inhibitor is dapagliflozin.

26. The pharmaceutical composition of any one of claims 21 to 25, for use in the method of any one of claims 10 to 20.

27. A kit comprising:
a first pharmaceutical composition comprising an RXFP1 modulator selected from: Compound 1, Compound 2, Compound 3, Compound 4, Compound 5 and Compound 6, or a pharmaceutically acceptable salt thereof, and a pharmaceutically acceptable excipient; and
a second pharmaceutical composition comprising an SGLT2 inhibitor and a pharmaceutically acceptable excipient.

28. The kit of claim 27, wherein the RXFP1 modulator is Compound 1 or a pharmaceutically acceptable salt thereof.

29. The kit of claim 27 or 28, wherein the SGLT2 inhibitor is selected from dapagliflozin, canagliflozin, empagliflozin, ertugliflozin, ipragliflozin, remogliflozin etabonate, sergliflozin etabonate, sotagliflozin and tofogliflozin, or a pharmaceutically acceptable salt thereof.

30. The kit of any one of claims 27 to 29, wherein the SGLT2 inhibitor is dapagliflozin.

31. The kit of any one of claims 27 to 30, wherein the RXFP1 modulator is Compound 1 or a pharmaceutically acceptable salt thereof, and the SGLT2 inhibitor is dapagliflozin.

32. The kit of any one of claims 27 to 31, for use in the method of any one of claims 10 to 20.
